(19) United States
(12) Patent Application Publication
(10) Pub. No.: US 2021/0164984 A1
(43) Pub. Date: Jun. 3, 2021

(54) METHODS FOR PREDICTING OUTCOME AND TREATMENT OF PATIENTS SUFFERING FROM PROSTATE CANCER OR BREAST CANCER

(71) Applicants: INSERM (Institut National de la Santé et de la Recherche Médicale), Paris (FR); Université de Paris, Paris (FR); Commissariat à l'Énergie Atomique et aux Énergies Alternatives (CEA), Paris (FR); Université Paris-Saclay, Saint Aubin (FR)

(72) Inventors: Claire MAGNON, Fontenay Aux Roses (FR); Paul-Henri ROMEO, Fontenay Aux Roses (FR)

(21) Appl. No.: 17/047,269

(22) PCT Filed: Apr. 15, 2019
(86) PCT No.: PCT/EP2019/059698

§ 371 Date: Oct. 13, 2020

(30) Foreign Application Priority Data
Apr. 13, 2018 (EP) 18305460.0

(51) Int. Cl. G01N 33/574

(52) U.S. Cl. CPC G01N 33/57434; G01N 33/57415; G01N 2496/00; G01N 2800/52; G01N 33/57484

(57) ABSTRACT

The invention relates to methods for predicting the outcome of a patient suffering from prostate cancer or breast cancer and methods for the treatment of prostate cancer or breast cancer. The inventors show that Doublecortin-expressing (DCX⁺) neural precursors from the central nervous system (CNS) enter the bloodstream, infiltrate prostate tumours and metastasis and differentiate into neo-neurons that contribute to tumour development. In human primary prostate tumours and transgenic mouse cancer tissues, the density of DCX⁺ neural progenitors is strongly associated with tumour aggressiveness, invasion and recurrence. In transgenic cancer mice, oscillations of DCX⁺ neural stem cells in the subventricular zone (SVZ), a neurogenic area of the CNS, were associated with egress of DCX⁺ cells from the SVZ to the bloodstream. These cells then reach the tumour where they initiate neurogenesis. Selective genetic depletion of DCX⁺ cells in mice inhibits the early phases of prostate cancer development, whereas ortho topic transplantation of DCX⁺ cells purified from prostate tumour or brain tissues promotes tumour growth and cancer cell dissemination. These results unveil a unique crosstalk between the CNS and the tumour that drives a process of neurogenesis necessary for prostate cancer development, and indicate a novel neural element of the tumour microenvironment as a potential target for cancer treatment. Thus, the invention relates to a method for predicting the outcome of a patient suffering from prostate cancer and compound targeting DCX⁺ neural progenitor cells for use in the treatment of prostate cancer.

### FIELD OF THE INVENTION

The present invention relates to methods for the diagnosis of patients suffering from prostate cancer, breast cancer or metastatic tumour. The present invention also relates to methods and pharmaceutical compositions for treatment of prostate cancer, breast cancer or metastatic tumour.

### BACKGROUND OF THE INVENTION:

The parallel between embryonic and tumour development suggests conserved regulatory pathways by normal and tumour microenvironments. As the embryo needs neurogenesis and angiogenesis to develop, primary tumour initiation and metastasis require the development of a nervous and a vascular network (1). Neurogenesis involves de novo production of functional neurons from neural precursors, and occurs throughout life in two neurogenic regions of the central nervous system (CNS), the subventricular zone (SVZ) that generates interneurons in the olfactory bulbs (OB), and the dentate gyms (DG) in the hippocampus (2). After brain injury, a functional wound-healing response occurs leading to a slight increase in the proliferation of neural progenitors and a migration of newborn neurons to injury sites (3). In cancer, numerous studies have clearly shown that newly formed nerve fibres infiltrate and expand to control tumorigenesis and metastasis (4-10). Neurotrophic factors are expressed and released within the tumour microenvironment (TME) to promote axonal outgrowth from neo- or pre-existing nerves, thereby building up nerve networks that generate neural signaling in tumours (4,7,8,11). In healthy prostate, sympathetic innervation can regenerate after injury, indicating a process of axonogenesis without any evidence of newly formed neurons (12), whereas in prostate cancer, the number of neurons per ganglia is increased, suggesting that a potential process of neurogenesis occurs in tumours and that this process supports tumour development and progression (13).

The present invention uncovers a novel type of crosstalk between CNS and prostate tumours or breast tumours, as it reveals a unique migration of central neural precursors that nurture tumour development. The present invention shows how a tumour could have a dialogue with the CNS to recruit cells that are required for its growth and dissemination.

Altogether, these results lead to novel avenues to diagnose and monitor cancer development and novel therapies targeting neural progenitors in the blood or tumour microenvironment.

### SUMMARY OF THE INVENTION:

The present invention relates to methods for the diagnosis of patients suffering from prostate cancer or breast cancer or metastatic tumour. The present invention also relates to methods and pharmaceutical compositions for the treatment of prostate cancer or breast cancer or metastatic tumour. In particular, the invention is defined by the claims.

### DETAILED DESCRIPTION OF THE INVENTION:

The development of autonomic nerve fibres in the tumour microenvironment is a pivotal event that regulates prostate cancer initiation and dissemination, but how nerves emerge in tumours is presently unknown. The inventors show that Doublecortin-expressing (DCX⁺) neural precursors from the central nervous system (CNS) enter the bloodstream, infiltrate prostate tumours and differentiate into neo-neurons that contribute to tumour development. In human primary prostate tumours and transgenic mouse cancer tissues, the density of DCX⁺ neural progenitors is strongly associated with tumour aggressiveness, invasion and recurrence. In transgenic cancer mice, oscillations of DCX⁺ neural stem cells in the subventricular zone (SVZ), a neurogenic area of the CNS, were associated with egress of DCX⁺ cells from the SVZ to the bloodstream. These cells then reach the tumour where they initiate neurogenesis. In accordance, the DCX⁺ progenitors in prostate tumour can differentiate into neurons ex vivo and build up a tumour-associated neural network in vivo. Selective genetic depletion of DCX⁺ cells in mice inhibits the early phases of prostate cancer development, whereas orthotopic transplantation of DCX⁺ cells purified from prostate tumour or brain tissues promotes tumour growth and cancer cell dissemination. These results unveil a unique crosstalk between the CNS and the tumour that drives a process of neurogenesis necessary for prostate cancer or breast cancer development, and indicate a novel neural element of the tumour microenvironment as a potential target for cancer treatment.

Accordingly, the present invention relates to a method for predicting the outcome of a patient suffering from prostate cancer or breast cancer or metastatic tumour comprising the steps of:
i) determining the quantity of DCX⁺ neural progenitor cells in a biological sample obtained from the subject,
ii) comparing the quantity determined at step i) with their corresponding predetermined reference value, and

iii) detecting differential between the quantity determined at step i) with the predetermined reference value will indicate the outcome of the patient.

As used herein, the term “patient” denotes a mammal. Typically, a patient according to the invention refers to any patient (particularly human) afflicted with prostate cancer or breast cancer or metastatic tumour. The term “patient” also refers to any patient afflicted with prostate cancer or breast cancer or metastatic tumour receiving anti-cancer therapy. The term “patient” also refers to any patient afflicted with prostate cancer following surgical prostate cancer resection or to any patient afflicted with breast cancer following surgical breast cancer resection.

As used herein, the term “prostate cancer” has its general meaning in the art and refers to prostate cancer such as revised in the World Health Organisation Classification. The term “prostate cancer” also refers to any type of prostate cancer and selected from the group of: Malignant neoplasm of prostate (C61); low and high grade dysplasia of prostate (N42.3, D07.5); Benign neoplasm of prostate (D21.1); Neoplasm of uncertain or unknown behavior of prostate (D40); localized prostate cancer; advanced prostate cancer; locally advanced prostate cancer; metastatic prostate cancer; hormone-sensitive prostate cancer (HSPCs); castration-resistant (CRPCs) prostate cancer.

In one embodiment, the prostate cancer is a metastatic prostate cancer.

As used herein, the term “breast cancer” has its general meaning in the art and refers to breast cancer such as revised in the World Health Organisation Classification. Additionally, the term “cancer breast” includes all types of breast cancer at all stages of progression. The earliest stage breast cancers are called stage 0 (a pre-cancerous condition, either ductal carcinoma in situ or lobular carcinoma in situ), and then range from stage I through IV. In stage IV of breast cancer, also known as metastatic breast cancer, the cancer has spread beyond the breast and regional lymph nodes. The staging system most often used for breast cancer is the American Joint Committee on Cancer (AJCC) TNM system, which is based on the size of the tumor, the spread to the lymph nodes in the armpits, and whether the tumour has metastasized.

In one embodiment, the breast cancer is a metastatic breast cancer.

As used herein, the term “metastatic cancer” refers to cancer cells which break away from where they first formed and travel through the blood or lymph system to form new tumors (called metastatic tumor or metastasis) in other parts of the body.

The term “biological sample” refers to any biological sample derived from the patient such as blood sample, biopsy sample, prostate cancer sample, breast cancer sample, tumour tissue sample, stroma of the tumour tissue, cerebrospinal fluid or subventricular zone (SVZ).

As used herein, the term “tumor tissue sample” has its general meaning in the art and encompasses pieces or slices of tissue that have been removed including following a surgical tumor resection. The tumor tissue sample can be subjected to a variety of well-known post-collection preparative and storage techniques (e.g., fixation, storage, freezing, etc.) prior to determining the cell quantities. Typically, the tumor tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (IHC) (using an IHC automate such as BenchMark® XT or Autostainer Dako, for obtaining stained slides).

As used herein, the term “Doublecortin” or “DCX” has its general meaning in the art and refers to a classical marker of neural precursors which are located in developing and adult neurogenic regions of the CNS (14-16).

As used herein, the term “DCX⁺ neural progenitor cells” has its general meaning in the art and refers to Doublecortin-expressing (DCX⁺) neural precursors from the central nervous system (CNS). The term “DCX⁺ neural progenitor cells” also refers to DCX⁺ neural precursor cells. The term “DCX⁺ neural precursor cells” also refers to DCX⁺ neural stem cells. According to the present invention, the term “DCX⁺ neural progenitor cells” refers to Doublecortin-expressing (DCX⁺) neural precursors from the central nervous system (CNS) that infiltrate prostate tumours or breast tumours and differentiate into neo-neurons that contribute to tumour development. The term “DCX⁺ neural progenitor cells” also refers to neural progenitor cells characterized by expressing at least one of the neural markers selected from the group consisting of Doublecortin (DCX), Polysialylated-neural cell adhesion molecule (PSA-NCAM) (17), Internexin (INA) (18,19), Sca-1, CD24, EGFR, or Nestin.

The term “DCX⁺ neural progenitor cells” in the prostate tumour or breast tumour or metastatic tumour also refers to GFAP⁻ GLAST⁻ CD45⁻ CD326⁻ CD49f⁻ CD31⁻ TER119⁻ Sca-1^{hi} CD24^{int} EGFR^{hi} DCX⁺ PSANCAM^{lo} cells. The term “DCX⁺ neural progenitor cells” also refers to GFAP⁻ GLAST⁻ CD45⁻ CD326⁻ CD49f⁻ CD31⁻ TER119⁻ Sca-1^{lo} CD24⁺ EGFR^{Int} DCX⁺ PSANCAM^{Int} cells. The term “DCX⁺ neural progenitor cells” also refers to GFAP⁻ GLAST⁻ CD45⁻ CD326⁻ CD49f⁻ CD31⁻ TER119⁻ Sca-1⁻ CD24^{int} EDGR^{int} DCX⁺ PSANCAM⁻ cells.

The term “DCX⁺ neural progenitor cells” in the blood also refers to CD45⁻ DCX⁺Sca-1⁻ PSANCAM^{-/lo} cells.

The term “DCX⁺ neural progenitor cells” in the subventricular zone (SVZ) also refers to GFAP⁺ GLAST⁺ CD45⁻ CD326⁻ CD49f⁻ CD31⁻ TER119⁻ Sca-1⁻ CD24⁻ EGFR⁻ DCX⁺ PSANCAM⁻ cells. The term “DCX⁺ neural progenitor cells” in the SVZ also refers to GFAP⁺ GLAST⁺ CD45⁻ CD326⁻ CD49f⁻ CD31⁻ TER119⁻ Sca-1^{-/lo} CD24⁺ EGFR^{-/lo} DCX⁺ PSANCAM⁺ cells. The term “DCX⁺ neural progenitor cells” in the SVZ also refers to GFAP⁺ GLAST⁺ CD45⁻ CD326⁻ CD49f⁻ CD31⁻ TER119⁻ Sea-1^{lo} CD24⁺ EGFR⁺ DCX⁺ PSANCAM^{int} cells.

As used herein, the term “quantity of DCX⁺ neural progenitor cells” has its general meaning in the art and refers to the number of DCX⁺ neural progenitor cells. The term “quantity of DCX⁺ neural progenitor cells” also refers to the density of DCX ⁺neural progenitor cells. The term “quantity of DCX⁺ neural progenitor cells” also refers to the percentage of DCX+ neural progenitor cells.

In some embodiment, the present invention relates to a method for predicting the outcome of a patient suffering from prostate cancer or breast cancer or metastatic tumour comprising the steps of:
i) determining the quantity of DCX⁺ neural progenitor cells in a biological sample obtained from the subject,
ii) comparing the quantity determined at step i) with their corresponding predetermined reference value, and
iii) concluding that the patient has a good prognosis when the level determined at step i) is lower than the predetermined reference value or concluding that the patient has a poor prognosis when the level determined at step i) is higher than the predetermined reference value.

As used herein, the term “Good Prognosis” refers to a patient afflicted with prostate cancer or breast cancer that is likely to not present aggressiveness and/or invasiveness of prostate cancer or breast cancer, and/or that is likely to not present recurrence of prostate cancer or breast cancer, and/or prostate cancer or breast cancer relapse, and/or that is likely to present a high overall survival (OS), event-free survival (EFS), metastasis-free survival (MFS), and/or Recurrence-free survival (RFS).

As used herein, the term “Poor Prognosis” or “Bad Prognosis” refers to a patient afflicted with prostate cancer or breast cancer that is likely to present aggressiveness and/or invasiveness of prostate cancer or breast cancer, and/or that is likely to present recurrence of prostate cancer or breast cancer and/or prostate cancer relapse or breast cancer relapse, and/or that is likely to present a short overall survival (OS), event-free survival (EFS), metastasis-free survival (MFS), and/or Recurrence-free survival (RFS).

As used herein, the “reference value” refers to a threshold value or a cut-off value. The setting of a single “reference value” thus allows discrimination between a poor and a good prognosis with respect to the aggressiveness, invasiveness and/or recurrence of prostate cancer or breast cancer or metastatic tumour, cancer relapse and/or overall survival (OS) for a patient. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Particularly, the person skilled in the art may compare the quantity (obtained according to the method of the invention) with a defined threshold value. In one embodiment of the present invention, the threshold value is derived from the quantity (or ratio, or score) determined in a biological sample derived from one or more patients having prostate cancer or breast cancer or metastatic tumour. Furthermore, retrospective measurement of the quantity (or ratio, or scores) in properly banked historical patient samples may be used in establishing these threshold values.

Predetermined reference values used for comparison may comprise “cut-off” or “threshold” values that may be determined as described herein. Each reference (“cut-off”) value may be predetermined by carrying out a method comprising the steps of

a) providing a collection of samples (such as tumour, blood) from patients suffering of prostate cancer or breast cancer or metastatic tumour;

b) determining the quantity of DCX⁺ neural progenitor cells for each sample contained in the collection provided at step a);

c) ranking the tumour samples according to said quantity of cells;

d) classifying said samples in pairs of subsets of increasing, respectively decreasing, number of members ranked according to their quantity of cells,

e) providing, for each sample provided at step a), information relating to the actual clinical outcome for the corresponding prostate cancer or breast cancer or metastatic tumour patient (i.e. the duration of the event-free survival (EFS), metastasis-free survival (MFS) or the overall survival (OS) or both);

f) for each pair of subsets of samples, obtaining a Kaplan Meier percentage of survival curve;

g) for each pair of subsets of samples calculating the statistical significance (p value) between both subsets;

h) selecting as reference value for the quantity of cells, the value of the quantity of cells for which the p value is the smallest.

For example, the quantity of DCX⁺ neural progenitor cells has been assessed for 100 cancer samples of 100 patients. The 100 samples are ranked according to their quantity of cells. Sample 1 has the best quantity of cells and sample 100 has the worst quantity of cells. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding prostate cancer or breast cancer or metastatic tumour patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the quantity of cells corresponding to the boundary between both subsets for which the p value is minimum is considered as the reference value. It should be noted that the reference value is not necessarily the median value of quantities of cells.

In routine work, the reference value (cut-off value) may be used in the present method to discriminate prostate cancer or breast cancer or metastatic tumour samples and therefore the corresponding patients.

Kaplan-Meier curves of percentage of survival as a function of time are commonly to measure the fraction of patients living for a certain amount of time after treatment and are well known by the man skilled in the art.

The man skilled in the art also understands that the same technique of assessment of the quantity of cells should of course be used for obtaining the reference value and thereafter for assessment of the quantity of cells of a patient subjected to the method of the invention.

In a particular embodiment, the score may be generated by a computer program.

In some embodiments, the score may be generated by algorithm, wherein the algorithm is selected from Linear Discriminant Analysis (LDA), Topological Data Analysis (TDA), Neural Networks, Support Vector Machine (SVM) algorithm and Random Forests algorithm (RF).selected from Linear Discriminant Analysis (LDA), Topological Data Analysis (TDA), Neural Networks, Support Vector Machine (SVM) algorithm and Random Forests algorithm (RF).

In some embodiments, the score may be generated by a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set.

In some embodiments, the score is generated by a computer program.

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In one embodiment, the reference value may correspond to the quantity of DCX⁺ neural progenitor cells determined in a sample associated with a patient having a good prognosis. Accordingly, a higher quantity of DCX⁺ neural progenitor cells than the reference value is indicative of a patient having poor prognosis, and a lower or equal quantity of DCX⁺ neural progenitor cells than the reference value is indicative of a patient having a good prognosis.

In another embodiment, the reference value may correspond to the quantity of DCX⁺ neural progenitor cells determined in a sample associated with a patient having a poor prognosis. Accordingly, a higher or equal quantity of DCX⁺ neural progenitor cells than the reference value is indicative of a patient having poor prognosis, and a lower quantity of DCX⁺ neural progenitor cells than the reference value is indicative of a patient having good prognosis.

In some embodiments, the method of the present invention is particularly suitable for predicting aggressiveness, invasiveness, and/or recurrence of prostate cancer or breast cancer of the patient.

Accordingly, the present invention also relates to a method for predicting the outcome of a patient suffering from prostate cancer or breast cancer or metastatic tumour comprising the steps of:
i) determining the quantity of DCX⁺ neural progenitor cells in a biological sample obtained from the subject,
ii) comparing the quantity determined at step i) with their corresponding predetermined reference value, and
iii) concluding that the patient has a non-aggressive, a non-invasive, and/or a non-recurrent prostate cancer or breast cancer or metastatic tumour when the level determined at step i) is lower than the predetermined reference value or concluding that the patient has an aggressive, an invasive, and/or a recurrent prostate cancer or breast cancer or metastatic tumour when the level determined at step i) is higher than the predetermined reference value.

In some embodiments, the method of the present invention is particularly suitable for predicting the survival time of the patient.

Accordingly, the present invention also relates to a method for predicting the outcome of a patient suffering from prostate cancer or breast cancer or metastatic tumour comprising the steps of:
i) determining the quantity of DCX⁺ neural progenitor cells in a biological sample obtained from the subject,
ii) comparing the quantity determined at step i) with their corresponding predetermined reference value, and
iii) concluding that the patient has a long survival time when the level determined at step i) is lower than the predetermined reference value or concluding that the patient has a short survival time when the level determined at step i) is higher than the predetermined reference value.

In some embodiments, the method of the present invention is particularly suitable for predicting the recurrence-free survival of the patient.

Accordingly, the present invention also relates to a method for predicting the outcome of a patient suffering from prostate cancer or breast cancer or metastatic tumour comprising the steps of:
i) determining the quantity of DCX⁺ neural progenitor cells in a biological sample obtained from the subject,
ii) comparing the quantity determined at step i) with their corresponding predetermined reference value, and
iii) concluding that the patient has a long recurrence-free survival when the level determined at step i) is lower than the predetermined reference value or concluding that the patient has a short recurrence-free survival when the level determined at step i) is higher than the predetermined reference value.

The quantity of DCX⁺ neural progenitor cells is determined by any well-known method in the art. In some embodiments, the quantity of DCX⁺ neural progenitor cells is determined such as described in the example. In some embodiments, the quantity of DCX⁺ neural progenitor cells is determined by flow cytometry. In some embodiments, the quantity of DCX⁺ neural progenitor cells is determined by IHC or immunofluorescence.

For example, the quantification of the DCX⁺ neural progenitor cells is performed by contacting the tumour tissue sample with a binding partner (e.g. an antibody) specific for a cell marker of said cells. Typically, the quantification of the DCX⁺ neural progenitor cells is performed by contacting the tissue tumour tissue sample with a binding partner (e.g. an antibody) specific for Doublecortin (DCX), Polysialylated-neural cell adhesion molecule (PSA-NCAM) (17), Internexin (INA) (18,19), Sca-1, CD24, EGFR or Nestin.

In some embodiments, the quantification of the DCX⁺ neural progenitor cells is performed by contacting the tissue tumour tissue sample with both binding partners (e.g. an antibody) specific for Doublecortin (DCX) and for Polysialylated-neural cell adhesion molecule (PSA-NCAM) (17), Internexin (INA) (18,19), Sca-1, CD24, or Nestin, respectively.

Typically, the quantity of the DCX⁺ neural progenitor cells is expressed as the number of these cells that are counted per one unit of surface area of tissue sample, e.g. as the number of cells that are counted per mm² of surface area of tumour tissue sample. In some embodiments, the quantity of the DCX⁺ neural progenitor cells may also consist of the percentage of the specific cells per total cells (set at 100%).

Immunohistochemistry typically includes the following steps i) fixing the tumor tissue sample with formalin, ii) embedding said tumour tissue sample in paraffin, iii) cutting said tumor tissue sample into sections for staining, iv) incubating said sections with the binding partner specific for the marker, v) rinsing said sections, vi) incubating said section with a secondary antibody typically biotinylated and vii) revealing the antigen-antibody complex typically with avidin-biotin-peroxidase complex. Accordingly, the tumour tissue sample is firstly incubated the binding partners. After washing, the labelled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 125I)and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled. The resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample, or the absolute number of cells positive for the maker of interest, or the surface of cells positive for the maker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantitate the presence of the specified biomarker (i.e. the marker). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms and tissue recognition pattern (e.g. Aperio Spectrum Software, Automated QUantitatative Analysis platform (AQUA® platform), or Tribvn with Ilastic and Calopix software), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the marker) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. For example, the amount can be quantified as an absolute number of cells positive for the maker of interest. In some embodiments, a score is given to the sample that is a numerical representation of the intensity or amount of the histochemical staining ofthe sample, and represents the quantity ofthe DCX+ neural progenitor cells (e.g., the marker) present in the sample. Optical density or percentage area values can be given a scaled score, for example on an integer scale. Thus, in some embodiments, the method of the present invention comprises the steps consisting in i) providing one or more immunostained slices of tissue section obtained by an automated slide-staining system by using a binding partner capable of selectively interacting with the marker (e.g. an antibody as above described), ii) proceeding to digitalisation of the slides of step i).by high resolution scan capture, iii) detecting the slice of tissue section on the digital picture iv) providing a size reference grid with uniformly distributed units having a same surface, said grid being adapted to the size of the tissue section to be analysed, and v) detecting, quantifying and measuring intensity or the absolute number of stained cells in each unit whereby the number or the density of cells stained of each unit is assessed.

In some embodiments, the quantification of the DCX⁺ neural progenitor cells is performed by flow cytometry or Fluorescence-activated cell sorting (FACS). In some embodiments, the quantification of the DCX⁺ neural progenitor cells is performed by flow cytometry such as described in the example.

In a further aspect, the method of the present invention is suitable for determining whether a patient is eligible or not to an anti-cancer treatment or an anti-cancer therapy. For example, when it is concluded that the patient has a poor prognosis then the physician can take the choice to administer the patient with an anti-cancer treatment. Typically, the treatment includes chemotherapy, radiotherapy, radioimmunotherapy and immunotherapy.

The term “anti-cancer treatment” or “anti-cancer therapy” has its general meaning in the art and refers to anti-cancer compounds used in anti-cancer therapy such as tyrosine kinase inhibitors, tyrosine kinase receptor (TKR) inhibitors, EGFR tyrosine kinase inhibitors, anti-EGFR compounds, anti-HER2 compounds, Vascular Endothelial Growth Factor Receptors (VEGFRs) pathway inhibitors, interferon therapy, alkylating agents, anti-metabolites, immunotherapeutic agents such as sipuleucel-T, Androgen deprivation therapy (ADT), Interferons (IFNs), Interleukins, beta blockers, muscarinic receptor antagonist, radiotherapeutic agents (such as Ra223, Pb212) and chemotherapeutic agents such as described below.

The term “tyrosine kinase inhibitor” or “TKI” has its general meaning in the art and refers to any of a variety of therapeutic agents or drugs such as compounds inhibiting tyrosine kinase, tyrosine kinase receptor inhibitors (TKRI), EGFR tyrosine kinase inhibitors, EGFR antagonists. The term “tyrosine kinase inhibitor” or “TKI” has its general meaning in the art and refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to Erlotinib, sunitinib (Sutent; SU11248), dasatinib (BMS-354825), PP2, BEZ235, saracatinib, gefitinib (Iressa), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more particularly at least one Phase II clinical, even more particularly at least one Phase III clinical trial, and most particularly approved by the FDA for at least one hematological or oncological indication. Examples of such inhibitors include, but are not limited to Erlotinib, Gefitinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

EGFR tyrosine kinase inhibitors as used herein include, but are not limited to compounds selected from the group consisting of but not limited to Erlotinib, lapatinib, Rociletinib (CO-1686), gefitinib, Dacomitinib (PF-00299804), Afatanib, Brigatinib (AP26113), WJTOG3405, NEJ002, AZD9291, HM61713, EGF816, ASP 8273, AC 0010. Examples of antibody EGFR inhibitors include Cetuximab, panitumumab, matuzumab, zalutumumab, nimotuzumab, necitumumab, Imgatuzumab (GA201, RO5083945), and ABT-806.

The term “beta blockers” has its general meaning in the art and refers to any of a variety of therapeutic agents or drugs such as compounds blocking the receptor sites for the endogenous catecholamines epinephrine (adrenaline) and norepinephrine (noradrenaline) on adrenergic beta receptors, of the sympathetic nervous system.

The term “muscarinic receptor antagonist” has its general meaning in the art and refers to a type of anticholinergic agents that blocks the activity of the muscarinic acetylcholine receptor.

The term “chemotherapeutic agent” has its general meaning in the art and refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; amino levulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11 ; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazo les, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

The term “anti-cancer treatment” or “anti-cancer therapy” also refers to targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs”, “molecularly targeted therapies”, “precision medicines”, or similar names. In some embodiments, the targeted therapy consists of administering the subject with a tyrosine kinase inhibitor as defined above.

The term “anti-cancer treatment” or “anti-cancer therapy” also refers to immunotherapeutic agent. The term “immunotherapeutic agent” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, immune checkpoint inhibitor, cytokines, cancer vaccines, monoclonal antibodies and non-cytokine adjuvants. Alternatively, the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), and IFN-beta (IFN-β). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include sargramostim. Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. In addition to having specific or non-specific targets, immunotherapeutic agents can be active, i.e. stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDL1 antibodies, anti-PLD2 antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference]. The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most particularly be the subject's own cells that were earlier isolated from a blood or tumor sample and activated (or “expanded”) in vitro.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules).

Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, PD-L2, LAG-3, TIM-3 and VISTA.

The term “anti-cancer treatment” or “anti-cancer therapy” also refers to BRAF inhibitors such as vemurafenib, dacarbazine, dabrafenib, BMS-908662, LGX818, PLX3603, RAF265, R05185426, GSK2118436 and compounds described in Morris and Kopetz, 2013.

The term “anti-cancer treatment” or “anti-cancer therapy” also refers to radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy such as Ra223 or Pb212. Such methods can optionally further comprise the administration of one or more additional cancer therapies, such as, but not limited to, chemotherapies, and/or another radiotherapy.

As used herein, the term “radiotherapy” for “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In one embodiment, said active compounds may be contained in the same composition or administrated separately.

In a further aspect, the method of the present invention is suitable for determining whether a patient is eligible or not to a treatment with a compound targeting DCX⁺ neural progenitor cells. For example, when it is concluded that the patient has a poor prognosis then the physician can take the choice to administer the patient with a compound targeting DCX⁺ neural progenitor cells.

In a further aspect, the present invention relates to a compound targeting DCX⁺ neural progenitor cells for use in the treatment of prostate cancer or breast cancer or metastatic tumour in a patient in need thereof.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The term “compound targeting DCX⁺ neural progenitor cells” refers to any compound selectively targeting DCX⁺ neural progenitor cells such as DCX inhibitor, CD24 inhibitor, EGFR inhibitor, Nestin inhibitor, Sca-1 modulator and PSANCAM modulator.

Accordingly, the present invention also relates to a compound selected from the group consisting of DCX inhibitor, CD24 inhibitor, EGFR inhibitor, Nestin inhibitor, Sca-1 modulator and PSANCAM modulator for use in the treatment of prostate cancer or breast cancer or metastatic tumour in a subject in need thereof.

Typically, 1, 2, 3, 4, 5 or 6 compound selected from the group consisting of DCX inhibitor, CD24 inhibitor, EGFR inhibitor, Nestin inhibitor, Sca-1 modulator and PSANCAM modulator is used according to the invention.

In some embodiments, the present invention relates to a DCX inhibitor for use according to the invention.

In some embodiments, the present invention relates to a CD24 inhibitor for use according to the invention.

In some embodiments, the present invention relates to a EGFR inhibitor for use according to the invention.

In some embodiments, the present invention relates to a Nestin inhibitor for use according to the invention.

In some embodiments, the present invention relates to a Sca-1 modulator for use according to the invention.

In some embodiments, the present invention relates to a PSANCAM modulator for use according to the invention.

In some embodiments, the present invention relates to a DCX inhibitor and a CD24 inhibitor as a combined preparation for use according to the invention.

In some embodiments, the present invention relates to a DCX inhibitor, a CD24 inhibitor and EGFR inhibitor as a combined preparation for use according to the invention.

In some embodiments, the present invention relates to a DCX inhibitor, a CD24 inhibitor, EGFR inhibitor and Nestin inhibitor as a combined preparation for use according to the invention.

In some embodiments, the present invention relates to a DCX inhibitor, a CD24 inhibitor, EGFR inhibitor, Nestin inhibitor and a Sca-1 modulator as a combined preparation for use according to the invention.

In some embodiments, the present invention relates to a DCX inhibitor, a CD24 inhibitor, EGFR inhibitor, Nestin inhibitor, a Sca-1 modulator and PSANCAM modulator as a combined preparation for use according to the invention.

In some embodiments, the present invention relates to a CD24 inhibitor and EGFR inhibitor as a combined preparation for use according to the invention.

In some embodiments, the present invention relates to a CD24 inhibitor, EGFR inhibitor, and Nestin inhibitor as a combined preparation for use according to the invention.

In some embodiments, the present invention relates to a CD24 inhibitor, EGFR inhibitor, Nestin inhibitor and a Sca-1 modulator as a combined preparation for use according to the invention.

In some embodiments, the present invention relates to a CD24 inhibitor, EGFR inhibitor, Nestin inhibitor, a Sca-1 modulator and PSABCAM modulator as a combined preparation for use according to the invention.

In some embodiments, the present invention relates to EGFR inhibitor, Nestin inhibitor as a combined preparation for use according to the invention.

In some embodiments, the present invention relates to EGFR inhibitor, Nestin inhibitor, and a Sca-1 modulator as a combined preparation for use according to the invention.

In some embodiments, the present invention relates to EGFR inhibitor, Nestin inhibitor, a Sca-1 modulator and PSABCAM modulator as a combined preparation for use according to the invention.

In some embodiments, the present invention relates to Nestin inhibitor and a Sca-1 modulator as a combined preparation for use according to the invention.

In some embodiments, the present invention relates to Nestin inhibitor, a Sca-1 modulator and PSABCAM modulator as a combined preparation for use according to the invention.

The term “modulator” has its general meaning in the art and refers to a target inhibitor or activator. The term “modulator” also refers to a compound that increase or decrease the expression of a specific gene.

The term “activator” has its general meaning in the art and refers to any compound that can directly or indirectly stimulate the signal transduction cascade related to the target (Sca-1 and/or PSANCAM). The term “activator” also refers to a compound that selectively activates the target. Typically, a Sca-1 activator and/or PSANCAM activator is a small organic molecule, a peptide, a modified Sca-1 and/or PSANCAM or an activator of Sca-1 and/or PSANCAM expression.

An “activator of expression” refers to a natural or synthetic compound that has a biological effect to activate the expression of a gene.

The term “inhibitor” has its general meaning in the art and refers to a compound that selectively blocks or inactivates the target (DCX, CD24, EGFR, Nestin, Sca-1 and/or PSANCAM), to specifically kill the neural precursors (for example, antibodies conjugated with Ra223 or Pb²¹²) or inhibit their differentiation in neo-neurons. The term “inhibitor” also refers to a compound that selectively blocks the binding of the target to its substrate. The term “inhibitor” also refers to a compound able to prevent the action of the target for example by inhibiting the target controls of downstream effectors such as inhibiting the activation of the target pathway signaling. As used herein, the term “selectively blocks or inactivates” refers to a compound that preferentially binds to and blocks or inactivates the target with a greater affinity and potency, respectively, than its interaction with the other sub-types of the target family. Compounds that block or inactivate the target, but that may also block or inactivate other target sub-types, as partial or full inhibitors, are contemplated. The term “inhibitor” also refers to a compound that inhibits the target expression. Typically, an inhibitor is a small organic molecule, a polypeptide, an aptamer, an antibody, an oligonucleotide or a ribozyme.

Tests and assays for determining whether a compound is an inhibitor are well known by the skilled person in the art such as described in Pastrana et al and Chapker et al (25; 26).

In another embodiment, the target inhibitor of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against the target of the invention as above described, the skilled man in the art can easily select those blocking or inactivating the target.

In another embodiment, the target inhibitor of the invention is an antibody (the term including “antibody portion”) directed against the target.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion ofthe antibody comprises a F(ab′)2 portion ofthe antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of the target. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in the target. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Particularly, techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc' and Fc regions, for example, are effectors of the complement cascade but are not involved in antigen binding. An antibody from which the pFc' region has been enzymatically cleaved, or which has been produced without the pFc' region, designated an F(ab')2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc' regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab') 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab')2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGl, IgG2, IgG3 and IgG4. In a particular embodiment, the inhibitor of the invention is a Human IgG4.

In some embodiments, the invention provides a multispecific antibody comprising a first antigen binding site from an antibody of the present invention directed against DCX, CD24, EGFR, Nestin, Sca-1 and/or PSANCAM and at least one second antigen binding site.

In some embodiments, the second antigen-binding site is used for recruiting a killing mechanism such as, for example, by binding an antigen on a human effector cell as a BiTE (Bispecific T-Cell engager) antibody which is a bispecific scFv2 directed against target antigen and CD3 on T cells described in U.S. Pat. No. 7,235,641, or by binding a cytotoxic agent or a second therapeutic agent. As used herein, the term “effector cell” refers to an immune cell which is involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, mast cells and granulocytes, such as neutrophils, eosinophils and basophils. Some effector cells express specific Fc receptors (FcRs) and carry out specific immune functions. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs.

In some embodiments, the second antigen-binding site binds a tissue-specific antigen, promoting localization of the bispecific antibody to a specific tissue.

In some embodiments, the second antigen-binding site binds to an antigen located on the same type of cell as the [DCX, CD24, EGFR, Nestin, Sca-1 and/or PSANCAM]-expressing cell, typically a tumor-associated antigen (TAA), but has a binding specificity different from that of the first antigen-binding site. Such multi- or bispecific antibodies can enhance the specificity of the tumor cell binding and/or engage multiple effector pathways. Exemplary TAAs include carcinoembryonic antigen (CEA), human telomerase reverse transcriptase (hTERT), prostate specific antigen (PSA), CT antigens (such as MAGE-B5, -B6, -C2, -C3, and D; Mage-12; CT10; NY-ESO-1, SSX-2, GAGE, BAGE, MAGE, and SAGE), mucin antigens (e.g., MUC1, mucin-CA125, etc.), ganglioside antigens, tyrosinase, gp75, c-Met, Marti, MelanA, MUM-1, MUM-2, MUM-3, HLA-B7, Ep-CAM or a cancer-associated integrin, such as αβ3 integrin. Alternatively, the second antigen-binding site binds to a different epitope of [DCX, CD24, EGFR, Nestin, Sca-1 and/or PSANCAM]. The second antigen-binding site may alternatively bind an angiogenic or neurotrophic factor or other cancer-associated growth factor, such as a vascular endothelial growth factor, a fibroblast growth factor, epidermal growth factor, angiogenin, NT-3, NT-4, NGF, BDNF or a receptor of any of these, particularly receptors associated with cancer progression.

In some embodiments, the second antigen-binding site is from a second antibody or ADC of the invention, such as the antibody of the present invention.

Exemplary formats for the multispecific antibody molecules of the invention include, but are not limited to (i) two antibodies cross-linked by chemical heteroconjugation, one with a specificity to [DCX, CD24, EGFR, Nestin, Sca-1 and/or PSANCAM] and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab')2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain”, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the bispecific antibody is obtained or obtainable via a controlled Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is the antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is the antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety. The following are exemplary embodiments of combinations of such assymetrical mutations, optionally wherein one or both Fc-regions are of the IgG1 isotype.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874, 541 and 6,015,695). The “Hamers patents” more particularly describe production of VHHs in bacterial hosts such asE. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. 6,838,254).

In some embodiments, the antibody or the bispecific antibody of the invention is linked or conjugated to agent such as chemotherapeutic agent, radioactive particle (such as Ra223 or Pb²¹²) or toxin.

Typically, the inhibitors according to the invention as described above are administered to the patient in a therapeutically effective amount.

By a “therapeutically effective amount” ofthe inhibitor ofthe present invention as above described is meant a sufficient amount of the inhibitor for treating cancer at a reasonable benefit/risk ratio applicable to any medical treatment. It will be understood, however, that the total daily usage of the inhibitors and compositions of the present invention will be decided by the attending physician within the scope of sound medical judgment. The specific therapeutically effective dose level for any particular patient will depend upon a variety of factors including the disorder being treated and the severity of the disorder; activity of the specific inhibitor employed; the specific composition employed, the age, body weight, general health, sex and diet of the patient; the time of administration, route of administration, and rate of excretion of the specific inhibitor employed; the duration of the treatment; drugs used in combination or coincidential with the specific inhibitor employed; and like factors well known in the medical arts. For example, it is well within the skill of the art to start doses of the inhibitor at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the inhibitor of the present invention for the symptomatic adjustment of the dosage to the patient to be treated. A medicament typically contains from about 0.01 mg to about 500 mg of the inhibitor of the present invention, particularly from 1 mg to about 100 mg of the inhibitor of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the compound according to the invention may be used in a concentration between 0.01 μM and 20 μM, particularly, the compound of the invention may be used in a concentration of 0.01, 0.05, 0.1, 0.5, 1.0, 2.5, 5.0, 10.0, 15.0, 20.0 μM.

According to the invention, the compound of the present invention is administered to the subject in the form of a pharmaceutical composition. Typically, the compound of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, the composition includes isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized agent of the present inventions into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the compound of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In some embodiments, the compounds of the present invention is administered in combination with an anti-cancer treatment.

In some embodiments, the compound of the present invention is administered simultaneously, sequentially or concomitantly with one or more therapeutic active agent such as anti-cancer therapy such as immunotherapeutic agent, chemotherapeutic agent or radiotherapeutic agent.

In one embodiment, said additional active compounds may be contained in the same composition or administrated separately.

In another embodiment, the pharmaceutical composition of the invention relates to combined preparation for simultaneous, separate or sequential use in the treatment of prostate cancer or breast cancer or metastatic tumour in a patient in need thereof.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The invention also provides kits comprising the compound of the invention. Kits containing the compound of the invention find use in therapeutic methods.

Thus, the invention also refers to a kit or device for performing the method of the present invention, comprising means for determining the level of DCX⁺ neural progenitor cells in a biological sample.

In one embodiment, the kit or device comprises means for determining at least one of the neural markers selected from the group consisting of Doublecortin (DCX), Polysialylated-neural cell adhesion molecule (PSA-NCAM), Internexin (INA), Sca-1, CD24, EGFR or Nestin.

In a further aspect, the present invention relates to a method for treating prostate cancer or breast cancer or metastatic tumour in a patient in need thereof, comprising administering to the patient a therapeutically effective amount of a compound targeting DCX⁺ neural progenitor cells.

In a further aspect, the present invention relates to a method for treating prostate cancer or breast cancer or metastatic tumour in a patient in need thereof, comprising administering to the patient a therapeutically effective amount of a compound selected from the group consisting of DCX inhibitor, CD24 inhibitor, EGFR inhibitor, Nestin inhibitor, Sca-1 modulator and/or PSANCAM modulator.

In a further aspect, the present invention relates to a method for treating prostate cancer or breast cancer or metastatic tumour in a patient in need thereof, comprising administering to the patient a therapeutically effective amount of 1,2,3,4,5 or 6 compounds selected from the group consisting of DCX inhibitor, CD24 inhibitor, EGFR inhibitor, Nestin inhibitor, Sca-1 modulator and PSANCAM modulator.

In some embodiment, the invention also refers to a method for treating prostate cancer or breast cancer or metastatic tumour in a patient in need thereof, comprising administering to the patient a therapeutically effective amount of a compound targeting DCX⁺ neural progenitor cells in combination with a classical treatment.

As used herein, the term “classical treatment” refers to chemotherapy, radiotherapy, radio immunotherapy and immunotherapy.

In a further aspect, the present invention relates to a method of screening a candidate compound for use as a drug for treating cancer in a patient in need thereof, wherein the method comprises the steps of:

providing a DCX, CD24, EGFR, Nestin, Sca-1 and/or PSANCAM, providing a cell, tissue sample or organism expressing a DCX, CD24, EGFR and/or Nestin, providing DCX⁺ neural progenitor cells,

providing a candidate compound such as a small organic molecule, a polypeptide, an aptamer, an antibody or an intra-antibody,

quantifying the DCX, CD24, EGFR, Nestin, Sca-1 and/or PSANCAM expression and/or activity,

and selecting positively candidate compounds that inhibit DCX, CD24 and/or Nestin expression and/or activity, and/or that modulate Sca-1 and/or PSANCAM expression and/or activity.

Methods for measuring DCX, CD24, EGFR, Nestin, Sca-1 and/or PSANCAM activity are well known in the art (25-26). For example, measuring the DCX, CD24, EGFR, Nestin, Sca-1 and/or PSANCAM activity involves determining a Ki on the DCX, CD24, EGFR, Nestin, Sca-1 and/or PSANCAM cloned and transfected in a stable manner into a CHO cell line, measuring DCX⁺ neural progenitor cells viability/survival, measuring cancer cell migration and invasion abilities, measuring cancer cell growth, measuring cancer cell proliferation in the present or absence of the candidate compound.

Tests and assays for screening and determining whether a candidate compound is a DCX, CD24, EGFR and/or Nestin inhibitor, Sca-1 modulator and/or PSANCAM modulator are well known in the art (25-26). In vitro and in vivo assays may be used to assess the potency and selectivity of the candidate compounds to inhibit DCX, CD24, EGFR and/or Nestin activity, or to modulate Sca-1 and/or PSANCAM activity.

Activities of the candidate compounds, their ability to bind DCX, CD24, EGFR, Nestin, Sca-1 and/or PSANCAM and their ability to inhibit DCX, CD24, EGFR and/or Nestin activity, modulate Sca-1 and/or PSANCAM activity may be tested using isolated cancer cell, cancer cell lines or CHO cell line cloned and transfected in a stable manner by the human DCX, CD24, EGFR, Nestin, Sca-1 and/or PSANCAM.

The invention will be further illustrated by the following figures and examples. However, these examples and figures should not be interpreted in any way as limiting the scope of the present invention.

### FIGURES

FIG. 1. Prognostic value of Doublecortin-expressing (DCX⁺) neural precursors infiltrating human prostate adenocarcinomas. A, B. Quantification of DCX⁺ cells in benign prostatic hyperplasia (BPH, n=15), low-risk (n=17) or high-risk (n=20) human prostate adenocarcinomas. A, For a patient, each bar represents the average number of DCX⁺ cells obtained from 10 fields per normal (white), cancer (black) or hyperplastic (striped) area, 860 z-stacks confocal images, field surface=0.15 mm². B. Average number of DCX⁺ cells per field in normal (white) and cancer (black) tissues of low-risk (Lo) or high-risk (Hi) patients and in BPH areas (striped) of the patients studied in A. C. Association between the number of DCX⁺ cells and the number of prostate areas moderately (1) or highly (2,3,4) invaded by tumor cells. D. Recurrence-free survival of patients with high (>20 DCX⁺ cells) and low (<20 DCX⁺ cells) number of DCX⁺ cells. Scale bar, 20 μm. Error bars indicate SE. **P<0.01, ***P<0.001, ****P<0.0001. Error bars indicate SEM.

FIG. 2. Early time point analysis of recurrence-free survival of high-risk prostate cancer patients. Early recurrence after radical prostatectomy is associated with high number of DCX⁺ cells (>20 DCX⁺ cells). P=0.0338, log-rank (Mantel-Cox). *P<0.05. Same cohort of patients, shown in FIG. 1D, but focused on high-risk tumours.

FIG. 3. Lin⁻ tdTom⁺ cells can home in Pten prostate, PyMT breast tumours and metastasis. A. Frequencies of Lin⁻ tdTom⁺ cells in Pten mice (n=5) or wild-type littermates (n=8). Lin⁻ tdTom⁺ cells are also found in metastasis. Frequencies of Lin⁻ tdTom⁺ cells at week 12-14 after PC-3luc-xenograft in tumour tissues (B. colon. C. liver. D. lung and E. lymph nodes) by comparison to healthy tissues (no xenograft). Data are mean + SEM. Student t-test (one-sided, no adjustment), *P<0.05, **P<0.01. F. After injection of a tdTomato-expressing lentiviral vector by stereotaxy into to the SVZ at week 5 after birth, Lin−tdTom+ cells are found at week 16 after birth in PyMT breast tumours tissues by comparison to wild-type prostate or mammary pad tissues.

### EXAMPLE

Material & Methods

Mouse Strains

Balb/c nu/nu (B6.Cg-Foxn1^{nu}) and cMyc mice (FVB-Tg(ARR2/Pbsn-MYC)7Key (21), called Hi-Myc hereafter) were obtained from Charles River laboratories and the National Cancer Institute, respectively.

Hi-Myc mice were intercrossed with C57BL/6-Gt(ROSA)26Sortm1 (EYFP)Cos or C57BL/6-Gt(ROSA)26Sortm1 (HBEGF)Awai/J mice which were previously crossed with Tg(DCX-cre/ERT2)1Mul mice to generate Cre^{ERT2}-inducible expression of the enhanced yellow fluorescent protein (EYFP) or simian Diphtheria Toxin Receptor (DTR; from simian Hbegf) under the control of a doublecortin (DCX) promoter (all obtained from the Jackson laboratory). The resulting offsprings DCX-Cre^{ERT2}/loxp-EYFP/Hi-Myc or DCX-Cre^{ERT2}/loxp-HBEGF/Hi-Myc express EYFP or DTR, respectively, in DCX-expressing cells after administration of tamoxifen to the animals. Cells expressing DTR can be ablated following diphtheria toxin administration. Respective controls were also generated by intercrossing the three strains.

Immunodeficient B6.Cg-Foxn1^{nu+/-} heterozygous nude mice were also intercrossed with Tg(DCX-cre/ERT2)1Mul bred with Gt(ROSA)26Sortm1(HBEGF)Awai/J to deplete cells that express DCX in nu/nu mice.

Pten transgenic mice were generated by a specific Pten deletion (Pten^{loxp/loxp}) in prostate epithelial cells under the control of the probasin promoter (ARR2Pbsn-Cre, PB-cre4). PyMT transgenic mice express the polyomavirus PyV middle T antigen specifically in mammary epithelial cells under the control of mouse mammary tumor virus (MMTV).

Animal Procedures

All in vivo experiments were approved by the Animal Care and Use Committee of CEA (Fontenay-aux-Roses, France) as referred to the authorisation 2015022617149597.

For DCX-Cre^{ERT2}-mediated recombination, tamoxifen was prepared in corn oil (100 mg/kg, twice a day for 5 consecutive days) and was injected by intraperitoneal injection (Sigma-Aldrich, Saint-Louis, Mich.). For DCX⁺ cell depletion experiments, diphtheria toxin (4 μg/kg, Sigma-Aldrich) was injected once daily, intraperitoneally, 48 h after the last tamoxifen injection for 3 consecutive days.

For experiments in transgenic model, DCX-Cre^{ERT2}/loxp-EYFP/Hi-Myc mice were injected with tamoxifen at different time points between week 3 and week 24 after birth, and sacrificed 2 weeks after the last injection of tamoxifen. For inducible genetic tracing experiments, DCX-Cre^{ERT2}/loxp-EYFP/Hi-Myc mice were injected at week 3 after birth and animals were euthanized at 8, 12, 16 and 20 weeks after birth. For histological analyses after DCX⁺ cell depletion, DCX-Cre^{ERT2}/loxp-HBEGF/Hi-Myc animals were injected with tamoxifen (at week 3, week 4 and week 8 or week 12 and week 16 after birth) and killed at week 20.

For orthotopic xenogeneic model, human prostate tumours were induced by orthotopic surgical implantations of 1×10⁵ PC-3luc cells into 6 weeks-old Balb/c nu/nu mice. Three weeks after cell injection, the animals were randomized into the different groups and received, orthotopically, a vehicle or the appropriate transplant of purified Lin⁻ EYFP⁺ cells isolated from prostate, OB or SVZ tissues. For DCX⁺ cell depletion experiments, 1×10⁵ PC-3luc cells were implanted orthotopically into tamoxifen-injected DCX-Cre^{ERT2}/loxp-HBEGF/ nu/nu mice (or control littermates), 2 days after the last injection of diphtheria toxin, with or without the co-transplantation of purified sub-populations of Lin⁻ EYFP⁺ cells isolated from DCX-Cre^{ERT2}/loxp-EYFP/Hi-Myc prostate tumours.

Microdissection of the SVZ, OB and DG from the Adult Mouse Brain

After euthanasia, the head of mice was cut off above the cervical spinal cord region, and a medial caudal-rostral cut was made to remove the skin of the head. The skull was peeled outward to expose the brain. The olfactory bulbs were first removed and collected in RPMI medium supplemented with 10% FBS (Life technologies, Carlsbad, Calif.). Then, the brain was rotated to expose its ventral surface and the SVZ was isolated, under a dissecting microscope, by making a first coronal cut at the level of the base of the optic chiasm and a second cut just before the hippocampus (data not shown), resulting to a coronal section from which the subventricular zone was harvested and collected in RPMI medium supplemented with 10% FBS as described by Guo et al., 2005. The hippocampus containing the dentate gyms was dissected from the remaining caudal part of the brain (37). The medial surface of cerebral hemisphere was placed side up to expose the medial side of the hippocampus. A needle-tip was inserted at the boundary of the dentate gyms and Ammon's horn and slipped superficially along the septo-temporal axis of the hippocampus to isolate the DG which was collected in RPMI medium supplemented with 10% FBS.

Dissociation of Brain and Prostate Tissues

Prior to be dissociated, prostate tissues were minced with a scalpel blade, and then placed into C tube for enzymatic dissociation. Brain and prostate tissues were enzymatically dissociated to single-cell suspensions into gentleMACS C tubes using the respective neural (T) or tumor tissue dissociation kits and the gentleMACS octo dissociator with heaters. The gentleMACS programs NTDK_1 or m_TDK_1 were applied, respectively, as recommended by the manufacturer (Miltenyi biotec, Bergisch Gladbach, Germany). After dissociation, the cell suspension was applied to a MACS smartstrainer (70 μm), centrifuged and resuspended in MACS running buffer.

Cell Culture

PC-3 cells stably transfected with the luciferase 2 gene under the control of human ubiquitin C promoter (Perkin Elmer, Waltham, Mass.), were grown in F12-Glutamax medium supplemented with 10% FBS, 1.5 g/l Bicarbonate sodium (Life technologies, Carlsbad, Calif.).

After enzymatic dissociation of OB and prostate cells from DCX-Cre^{ERT2}/loxp-EYFP/Hi-Myc mice, cells were washed, resuspended in MACS neuro medium (Miltenyi biotec) supplemented with basic fibroblast growth factor (bFGF, 20 ng/ml) and epidermal growth factor (EGF, 20 ng/ml; Preprotech, Rocky Hill, N.J.), and then 10⁶ cells were plated into one well of a 24-well tissue culture plate (Thermofisher scientific, Waltham, Mass.) that was previously coated with Poly-L-ornithine (10 μg/ml, Sigma-Aldrich, Saint-Louis, Mich.) and laminin (10 μg/ml, Corning life sciences, Corning, N.Y.). Half of the medium was replaced every 48 h for 1 week. Then, adherent cells were harvested and passed onto Poly-L-ornithine- and laminin-coated μ-slide 8-well (ibidi, Martinsried, Germany). Cells were incubated in MACS neuro medium supplemented with bFGF (20 ng/ml) and EGF (20 ng/ml) or brain-derived neurotrophic factor (BDNF, 1 μg/ml, Miltenyi biotec) and neurotrophin-3 (NT-3, 0.1 μg/ml; Miltenyi biotec). The medium was replaced every 48 h until neural differentiation of the cells.

Bioluminescence Imaging

In vivo and ex vivo bioluminescence imaging was performed and analysed using an IVIS imaging system 200 series (Xenogen, Caliper Life Sciences, Hopkinton, Mass.). Bioluminescent signal was induced by i.p. injection of D-luciferin (150 mg/kg in PBS) 7 min prior to in vivo imaging. For ex vivo imaging, D-luciferin (300 mg/kg) was injected 8 min prior to necropsy. Organs of interest were immersed in a solution of D-luciferin at 150 mg/ml (4).

Histology and Immunofluorescence For paraffin-embedded sections, human and mouse prostate tissues were previously fixed in formalin or 4% paraformaldehyde, respectively. Blocks were serially sectioned (thickness 5 μm) and H&E staining was performed using standard procedures. Prior to stain, sections were deparaffinized with xylene and rehydrated through graded alcohol washes followed by antigen retrieval in sodium citrate buffer following manufacturer recommendations (Vector laboratories, Burlingame, Calif.).

For frozen sections, mice were anesthetized with 4% isoflurane before receiving a lethal dose of pentobarbital (60 mg/ml). Animals were then fixed by cardiac perfusion with 0.9% NaCl followed by 4% ice-cold paraformaldehyde (PFA) in 0.01 M PBS. The brain and prostate were collected, post-fixed overnight in 4% PFA at +4° C. and transferred in a 12% sucrose solution in PBS before snap freezing and cryostat sectioning (thickness 12 μm; Leica, Wetzlar, Germany).

For immunofluoresecnce, nonspecific binding was blocked with goat serum in BSA solution, and sections were incubated overnight with mouse antibodies to DCX (Millipore, Billerica, Mass.), to PSA-NCAM (ABC scientific, Los Angeles, Calif.) or to pan-cytokeratine (Sigma-Aldrich), rabbit antibodies to MAP-2 (Millipore), to βIII-tubulin (Covance, Princeton, N.J.) or to α-Internexin (Millipore), and chicken antibodies to NF-H (Millipore) or to EYFP (Ayes labs, Tigard, Oreg.). Secondary staining was subsquently performed for 1 h at room temperature with the appropriate Alexafluor647-, 568-, 488-conjugated goat antibodies to mouse, rabbit or chicken IgG respectively (Life technologies, Carlsbad, Calif.).

For dye administration, fluorescein-tagged albumin (65 kDa, 2 mg diluted in 0.1 ml saline; Sigma-Aldrich) and TRITC-tagged dextran (4.4 kDa, 2 mg diluted in 0.1 ml saline, Sigma-Aldrich) were simultaneously IV and IP injected into 5-month-old Hi-Myc cancer mice under anesthesia. After a circulation period of one hour, mice were deeply anesthetized with isoflurane and pentobarbital and euthanized by transcardial perfusion with 0.9% NaCl followed by 4% ice-cold paraformaldehyde (PFA) in 0.01 M PBS. Brains were collected, post-fixed overnight in 4% PFA at +4° C. and transferred in a 12% sucrose solution in PBS before snap freezing and cryostat sectioning (thickness 14 μm; Leica).

Bright-field images of full Hi-Myc prostate sections were captured and collected with a Zeiss axioscan Z1 (Zeiss MicroImaging, Thornwood, N.Y.) equipped with an Hitachi HV-F202FCL color camera controlled by Zen microscope software.

Fluorescence images were captured and analyzed using a Leica TCS SP8 X confocal microscope equipped with White Light Laser, a PMT SP confocal detector coupled with a Leica hybrid detector (HyD) for super-sensitive confocal imaging (Leica, Wetzlar, Germany). Images were obtained as three dimensional (3D) stacks scanning through the whole thickness of the tissue controlled by LAS X 2.0.1.14392 software and analysed using high performance 3D imaging Volocity 6.3.1 software (Perkin Elmer, Waltham, Mass.).

Human Prostate Samples

Radical prostatectomies were obtained for staining after institutional review board approval at the department of pathology and biological resources platform at Henri Mondor hospital (Créteil, France; CPP n°16169). Human prostate tissues were collected, fixed in formalin and embedded in paraffin as part of routine care at Henri Mondor hospital. For each block, a section was stained with H&E to evaluate tissue viability, to localize normal areas among cancer, and to map the different Gleason grade areas. All patient had histologically confirmed and clinically localized prostate cancer or benign hyperplasia, and did not received prior treatment at the institution. Patient characteristics including age, preoperative PSA levels, date of surgery, pathological stages and Gleason score are shown in Table 1. PSA recurrence was defined as a single PSA value at >0.2 ng/ml, two values at 0.2 ng/ml, or secondary treatment for a rising PSA. Recurrence might be local or distant, although no metastasis has been documented thus far in this cohort of patients. Extraprostatic extension was defined as disease involving one or more of extracapsular, ganglion, or seminal vesicle extension and positive surgical margins. Quantification of DCX⁺ cells was conducted blind, without knowledge of clinical data, in prostate tumour or hyperplastic areas and in remaining normal prostate tissues surrounding cancer areas. For each marker defined above, the average of 10 representative fields (one field=0.15 mm²) was calculated from normal areas and for tumour grade captured as described above. A total of 1040 z-stack images were acquired and converted in 2D maximum projections that were analyzed with the Volocity software to quantify DCX⁺/DAPI⁺ cells per field (DAPI, 4′,6-diamidino-2-phenylindole).

TABLE 1
Clinical and Pathological characteristics of men
with benign hyperplasia or prostate cancer

| Patient characteristics | | | | Tumor characteristics | | | | |
|---|---|---|---|---|---|---|---|---|
| | Age | | PSA | Date of | Gleason | Pathological | #invaded | Recurrence |
| # | (years) | Risk | (ng/ml) | the surgery | score | stage | [symbol] | 0−/1+ |
| 1 | 67 | BPH | 17 | 2013 | — | — | — | — |
| 2 | 72 | BPH | 7.42 | 2013 | — | — | — | — |
| 3 | 58 | BPH | 1.42 | 2013 | — | — | — | — |
| 4 | 85 | BPH | 3.3 | 2013 | — | — | — | — |
| 5 | 63 | BPH | 4.5 | 2014 | — | — | — | — |
| 6 | 85 | BPH | 6 | 2013 | — | — | — | — |
| 7 | 72 | BPH | — | 2013 | — | — | — | — |
| 8 | 55 | BPH | 0.92 | 2013 | — | — | — | — |
| 9 | 71 | BPH | 5.37 | 2013 | — | — | — | — |
| 10 | 68 | BPH | 4.9 | 2013 | — | — | — | — |
| 11 | 62 | BPH | 27 | 2013 | — | — | — | — |
| 12 | 69 | BPH | 3.6 | 2013 | — | — | — | — |
| 13 | 63 | BPH | 6.6 | 2013 | — | — | — | — |
| 14 | 83 | BPH | — | 2014 | — | — | — | — |
| 15 | 76 | BPH | 6 | 2014 | — | — | — | — |
| 16 | 65 | Low | 6.25 | 2007 | 6 | T2c | 0 | 0 |
| 17 | 63 | Low | 5.31 | 2007 | 6 | T2b | 0 | 0 |
| 18 | 68 | Low | 5.75 | 2007 | 6 | T2c | 0 | 0 |
| 19 | 69 | Low | 6.4 | 2007 | 6 | T2a | 0 | 0 |
| 20 | 59 | Low | 5 | 2007 | 6 | T2a | 1 | 0 |
| 21 | 72 | Low | 10.23 | 2006 | 6 | T2c | 0 | 0 |
| 22 | 58 | Low | 6.09 | 2006 | 6 | T2c | 0 | 0 |
| 23 | 64 | Low | 3.9 | 2007 | 6 | T2c | 0 | 0 |
| 24 | 55 | Low | 4.34 | 2007 | 6 | T2c | 0 | 0 |
| 25 | 59 | Low | 3.83 | 2006 | 6 | T2c | 0 | 0 |
| 26 | 63 | Low | 5.3 | 2007 | 6 | T3a | 1 | 0 |
| 27 | 60 | Low | 4.2 | 2007 | 6 | T2c | 1 | 0 |
| 28 | 59 | Low | 1.7 | 2008 | 6 | T2b | 0 | 0 |
| 29 | 53 | Low | 4.6 | 2008 | 6 | T2c | 0 | 0 |
| 30 | 73 | Low | 6.37 | 2008 | 6 | T2a | 1 | 0 |
| 31 | 76 | Low | 4.54 | 2008 | 6 | T2c | 0 | 0 |
| 32 | 60 | Low | 3.5 | 2009 | 6 | T2c | 1 | 1 |
| 33 | 60 | High | 13 | 2006 | 7 | T2a | 0 | 0 |
| 34 | 66 | High | 9.98 | 2007 | 7 | T2a | 0 | 0 |
| 35 | 71 | High | 9.6 | 2006 | 8 | T3b | 2 | 0 |
| 36 | 71 | High | 3.9 | 2006 | 8 | T3a | 2 | 1 |
| 37 | 70 | High | 13.22 | 2006 | 8 | T3a | 2 | 1 |
| 38 | 63 | High | 10.32 | 2008 | 8 | T3a | 2 | 1 |
| 39 | 75 | High | 12.26 | 2008 | 8 | T4 | 4 | 1 |
| 40 | 70 | High | 8.7 | 2008 | 8 | T3b | 3 | 1 |
| 41 | 53 | High | 2.9 | 2009 | 8 | T3b | 2 | 1 |
| 42 | 60 | High | 8.91 | 2009 | 8 | T4 | 3 | 1 |
| 43 | 60 | High | 16 | 2009 | 8 | T3a | 1 | 1 |
| 44 | 66 | High | 15.95 | 2009 | 8 | T3a | 2 | 1 |
| 45 | 62 | High | 7.11 | 2007 | 9 | T3a | 1 | 0 |
| 46 | 70 | High | 45 | 2008 | 9 | T3b | 4 | 1 |
| 47 | 71 | High | 8.7 | 2008 | 9 | T3b | 4 | 0 |
| 48 | 50 | High | 12 | 2009 | 9 | T3b | 4 | 1 |
| 49 | 72 | High | 7.6 | 2007 | 9 | T3b | 2 | 0 |
| 50 | 74 | High | 4.5 | 2007 | 9 | T3b | 3 | 1 |
| 51 | 72 | High | 77.1 | 2006 | 9 | T4 | 2 | 0 |
| 52 | 68 | High | 17.55 | 2006 | 9 | T3a | 2 | 0 |

BPH, Benign prostate hyperplasia; Low, Low-risk tumour; High, High-risk tumour
Low- and High-risk prostate cancers were defined as Gleason score <7 and Gleason score ≥7, respectively [symbol]
indicates data missing or illegible when filed

Flow Cytometry

The brain and prostate of DCX-Cre^{ERT2}/loxp-EYFP/Hi-Myc mice were dissected and dissociated as described above. Cellularity of SVZ, OB, DG and prostate were manually counted with viability in trypan blue using a Neubauer chamber before flow analysis. Then cells were incubated for 10 min. with FcR blocking reagent (Miltenyi biotec). Subsequently, fluorochrome-conjugated monoclonal antibodies specific to mouse CD45 (clone 30F11), TER119 (clone Ter-119), CD31 (clone 390), CD326 (clone caa7-9G8), CD49f (clone REA518), Sca-1 (clone REA422), PSA-NCAM (clone 2-2B), CD24 (clone M1/69) were used for 30 min at the concentration recommended by the manufacturer (Miltenyi biotec). Cells were also incubated with biotinylated EGF complexed with BV785-streptavidin (Invitrogen by Thermo Fischer Scientific). Analyses of stained cell suspensions were performed on a 5-laser SORP LSR II (355/408/488/561/640; BD BioSciences, San Jose, Calif.) and data were analysed with FlowJo software (Tree Star, Ashland, Oreg.). Cell populations were purified using a Becton Dickinson SORP ARIA II.

RNA Extraction, RT-PCR and Q-PCR

Gene expression levels were analysed from RNA extracted, using the RLT solution (Qiagen, Calsbad, Calif.), from purified cells isolated from the 8-week-old brain or 4-month- and 12-month-old prostate of DCX-Cre^{ERT2}/loxp-EYFP/Hi-Myc mice by quantitative real-time PCR. Reverse transcriptase (Superscript VILO; Invitrogen) was performed in accordance with the manufacturer's instructions. qPCR was performed with Fast SYBR Green (ABI Applied biosystems by Thermo Fischer scientific). Expression of glyceraldehyde-3-phosphate dehydrogenase (GAPDH) was used as a standard.

RNA-sequencing

Lin⁻ EYFP⁺ cells were isolated from 8-week-old brain or 16-week-old prostate of DCX-Cre^{ERT2}/loxp-EYFP/Hi-Myc mice, and were collected in Qiazol (Qiagen). RNA was extracted using the miRNeasy microkit (Qiagen) and mRNA libraries were prepared using Smart-Seq v4 Ultra Low Input RNA (Takara, Otsu, Shiga). Briefly, cDNA was synthesized by using the locked nucleic acid (LNA) technology integrated with SMART (Switching Mechanism at 5′ End of RNA Template) technology. For each library, post RT-PCR cycle number was adjusted according to the number of cells. Libraries were individually adapted and indexed using the Illumina Nextera XT kit and then, were controlled on the Agilent bioanalyzer (Tapestation 2200, Agilent Technologies). Identical librairies were pooled before sequencing at an average read depth of 70 millions reads per sample. Final libraries were controlled on Tapestation 2200 (Agilent Technologies) and were quantified with fluorimetric intercalant. All RNA-seq libraries were sequenced using the Illumina Nextseq 500 with HighOutPut cartridge to generate about 2×400 millions of 75 bases reads.

Sequenced reads were trimmed using trimmomatic version 0.36 based on a quality threshold of 33. Reads were aligned on the Mus Musculus genome release GRCm38.p6 using ultrafast RNA-seq aligner STAR. Quantification of gene expressions was performed using HTSeq version 0.10 based on default parameters and transcriptomic analyses were performed with the EdgeR package. The GO term id #GO:0043005 and #GO:0030182 were selected to study a list of genes associated to neuron projection and neuron differentiation. Hierarchical clustering in the heatmap representations were generated based on the Ward 2 distance and the complete linkage method. Correlation analyses restricted to the selected lists of genes belonging to Gene Ontologies were done using the Spearman coefficient of correlation and the statistical significance was determined based on the associated P-value. Expression analysis of genes in ImmGen populations was performed using the ImmGen web portal (based on the MyGeneSet service). Statistical comparisons between SVZ, OB, or prostate samples and ImmGen populations were performed based on the Spearman coefficient of correlation restricted to the lists of 200 most expressed genes in each sample.

Stereotaxic Injection

TdTomato cDNA was cloned under the control of the CAG promoter in a lentiviral shuttle plasmid that contains a self-inactivating HIV-1-derived genome (38). Recombinant lentiviral particles were produced by transient transfection of HEK-293T cells, and tittered by ELISA quantification of the p24 capsid protein (HIV-1 p24 antigen ELISA, Gentaur, France). Prior to inject in the subventricular zone of 5-week-old mice, viral particles were diluted in Phosphate-Buffered Saline to a final concentration of 50 ng of p24. μl⁻¹ and delivered in a volume of 2 μl by stereotaxic injection at the following coordinates: 0.7 mm anterior and 1.3 mm lateral relative to bregma; and 2.5 mm below the skull surface. Viral solution was injected at a speed of 0.2 μl min⁻¹ and the injection cannula was left in place for 5 minutes before being slowly removed.

Statistical Analyses

For mouse analyses, all values are reported as means ± SEM. Statistical significance was assessed by a nonparametric unpaired Mann-Whitney test. Significance was set at P<0.05.

For patient studies, statistical analyses were performed using R software. Statistical comparisons of the different groups were performed using Wilcoxon rank-sum tests. The non-influence of patient ages on statistical comparisons was verified using regression analyses. Correlation between the number of DCX⁺ cells and the number of invaded zones was identified using the Spearman coefficient of correlation. Survival curves were modeled using Kaplan-Meier estimates. Statistical significance of the difference between survival curves was then assessed using the log-rank (Mantel-Cox) test. P-values lower than 0.05 were considered as significant.

Results

Stromal DCX+ Cells and Tumour Aggressiveness in Human

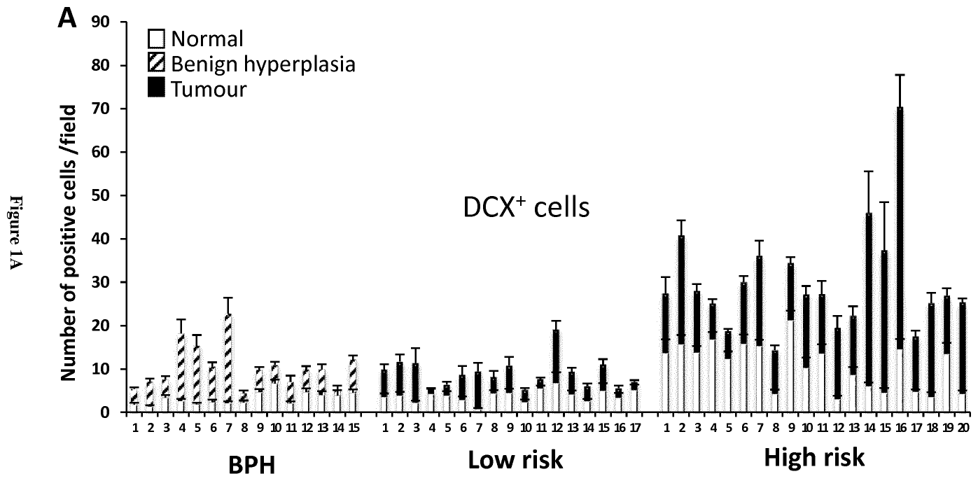

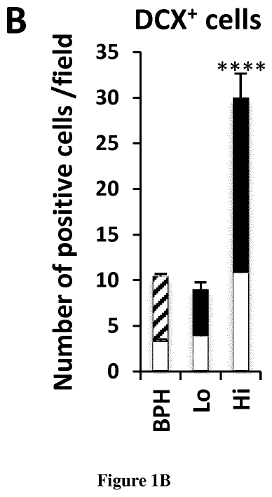

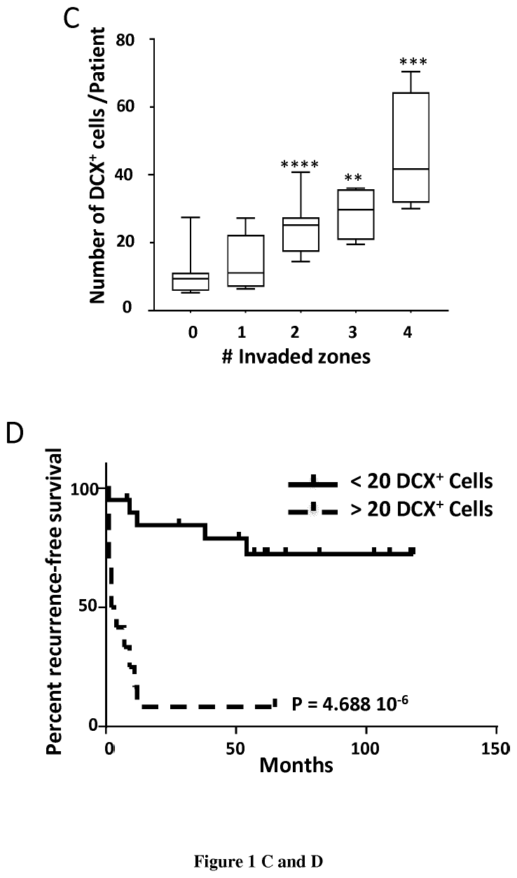

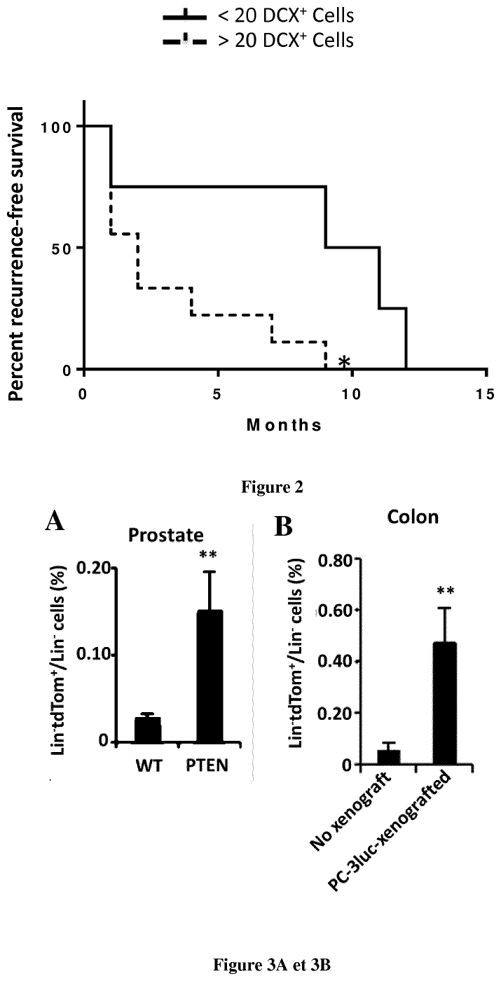

Doublecortin (DCX) is a classical marker of neural precursors which are located in developing and adult neurogenic regions of the CNS (14-16). Analysis of the stroma of human prostate primary tumours revealed DCX⁺ cells that also expressed specific markers of neural precursors (i.e. Polysialylated-neural cell adhesion molecule, PSA-NCAM (17); Internexin, INA (18,19); data not shown), but did not express markers of epithelial cells (Pancytokeratin, PanCK, data not shown) or mature nerve fibres (i.e. Neurofilament-Heavy, NF-H (20); data not shown). To assess a potential clinical relevance of the neo-development of a neuronal network in prostate cancer, we quantified DCX⁺ cells in low- and high-risk prostate cancer specimens from patients (37 treatment-naïve cancer patients versus 15 patients with benign prostate hyperplasia; Table 1). The density of DCX⁺ cells was highly associated with tumour aggressiveness (FIG. 1A, B; Table 2), and these cells were co-opted during malignancy to facilitate tumour development, invasion (Spearman's rank correlation coefficient=0.7797; P=7.97 10⁻⁹, FIG. 1C; Tables 3 and 4) and recurrence (P=4,688 10⁻⁶, log-rank (Mantel-Cox); FIG. 1D, FIG. 2) suggesting that DCX+ cells may have a role in controlling human prostate tumour development and progression.

TABLE 2
Statistical comparison between the number
of DCX⁺ cells for each type of human tissue

| Tissue type | P-value | 95% lower CI | 95% lower CI |
|---|---|---|---|
| Low (Normal) vs BPH | 0.1678 | −0.3000 | 1.8000 |
| High (Normal) vs BPH | 0.0000 | 2.7000 | 11.4000 |
| High (Normal) vs Low | 0.0001 | 2.3001 | 10.5000 |
| (Normal) | | | |
| Low (Tumour) vs BPH | 0.4499 | −3.5001 | 1.1999 |
| High (Tumour) vs BPH | 0.0001 | 6.1001 | 14.0000 |
| High (Tumour) vs Low | 0.0000 | 7.7999 | 14.8001 |
| (Tumour) | | | |

Abbreviations:
DCX, Doublecortine;
BPH, Benign prostate hyperplasia;
Low, Low-risk tumour;
High, High-risk tumour;
vs, versus;
CI, Confidence intervals

TABLE 3
Statistical comparison between the number of
DCX⁺ cells and the number of invaded zones

| # Invaded zones versus 0 zone | P-value | 95% lower CI |
|---|---|---|
| 1 zone | 0.3507 | −2.4 |
| 2 zones | 0.0001 | 8.3 |
| 3 zones | 0.0033 | 10.0 |
| 4 zones | 0.0008 | 20.6 |

Extra-prostatic extension was defined as a disease involving the invasion of one or more zones (extracapsular extention, seminar vesicle, ganglion and positive surgical margins)

TABLE 4
Correlation between the number of DCX+
cells and the number of invaded zones

| | Spearman | | | |
|---|---|---|---|---|
| Tissue type | coefficie [symbol] | P-value | 95% lower CI | 95% upper CI |
| Normal | 0.5825 | 1.55E−04 | 0.2870 | 0.8076 |
| Tumour | 0.7342 | 2.32E−07 | 0.5403 | 0.8540 |
| Combined | 0.7797 | 7.97E−09 | 0.5862 | 0.8832 |

[symbol]

indicates data missing or illegible when filed

DCX⁺ Neural Progenitors Initiate Tumour Neurogenesis

DCX⁺ cells were also present in the stroma of Hi-Myc mouse prostate cancer tissues (Hi-Myc mice express c-Myc specifically in prostate epithelial cells under the control of the probasin promoter (21); data not shown). Thus, to easily track, isolate and characterize DCX⁺ cells in Hi-Myc tumours, we generated triple-transgenic cancer mice (DCX promoter/enhancer driving tamoxifen-inducible Cre; DCX-Cre^{ERT2}/loxp-EYFP/Hi-Myc mice, data not shown). The presence of DCX:EYFP⁺ cells in the neurogenic areas (i.e. SVZ/OB and DG) of the brain showed the recombination efficiency (22) (data not shown). In prostate tumours, DCX:EYFP⁺ cells were found in the stromal compartment (data not shown) and characterized with the antigenic profile Lin⁻ (Lineage negative: CD45, TER119, CD31, CD326, CD49f) Sca-1⁺ (prostate stromal cells are defined as Lin⁻ Sca-1⁺) DCX:EYFP⁺ (hereafter called EYFP⁺) and PSA-NCAM⁺ (data not shown) (17,23,24). Lin⁻ EYFP⁺ cells were found in prostate tumours but not in prostate tissues from littermates without the c-Myc transgene (data not shown). This population expressed similar specific neural markers (i.e. PSA-NCAM, CD24, EGFR) than neural precursors isolated from the SVZ, OB and DG in the CNS (data not shown) (25,26). This result suggested the presence, in tumours, of neural precursors, which we decided to further characterize. Lin⁻ EYFP⁺ cells purified from tumours did not exhibit the activated neural stem cell signature (GFAP⁺ GLAST⁺ CD133⁺ EGFR⁺ MASH1^{+/-} Nestin^{+/-} CD24^{-/lo}) of Lin⁻ EYFP⁺ cells isolated from the brain, but rather, a neural progenitor signature (GFAP⁻ GLAST⁻ CD133^{-/lo} EGFR^{-/lo} Mash1^{-/lo} Nestin⁺ CD24⁺; data not shown) (25,26). Transcriptomic analysis of Lin⁻ EYFP⁺ cells purified from tumours did not show any similarities with gene expression profiling of immune or endothelial cells (data not shown) but showed neuron differentiation (data not shown) and neuron projection (data not shown) signatures that were significantly similar to the ones of Lin⁻ EYFP⁺ cells isolated from the SVZ or OB. In accordance with this phenotype, Lin⁻ EYFP⁺ progenitors, isolated from prostate tumours, could differentiate into newly born neuron ex vivo (data not shown). To explore the neurogenic capacity of the Lin⁻ EYFP⁺ progenitors in vivo (data not shown), and to determine the different stages of Lin⁻ EYFP⁺ neural progenitor cell lineage (data not shown), we performed inducible tissue-specific genetic tracing in prostate tumours. Activation of genetic recombination by tamoxifen at week3 after birth resulted in the presence of Lin⁻ EYFP⁺ progenitors in tumours but not in healthy tissues surrounding the prostate at week 8, 12 and 16 (Hi-Myc tumour areas begins to develop 12 weeks after birth, data not shown) and the emergence of EYFP⁺ INX⁺ nerve fibres from EYFP⁺ neuroblasts 8 months after birth, suggesting that neo-neurons arise and develop, in situ, in the TME from Lin⁻ EYFP⁺ neuroblasts (data not shown). To document this neuronal differentiation, 2 stromal sub-populations of EYFP⁺ progenitor cells were purified from tumours, based on the antigenic profile Lin⁻ EYFP⁺ Sca-1⁺ PSA-NCAM⁺ (hereafter called Sca^{lo} PSAN⁺ and Sca^{huh} PSAN⁺; data not shown). The Sca^{hi} PSAN⁺ population upregulated the expression of markers used for the isolation of activated precursors from the SVZ, such as Nestin, EGFR and MASH1, highlighting a potential activated state of these progenitors (such as transit-amplifying cells) (26). By contrast, the Sca^{lo} PSAN⁺ populations had lower neural marker expression, suggesting a less activated status, but exhibited expression of the Neuro-D1 transcription factor required for neuronal differentiation (27) (i.e neuroblasts (26); data not shown). The frequencies of the 2 sub-populations fluctuated over time, reflecting that different stages of differentiation occur along with tumour development (data not shown). These results suggested that activated Sca^{hi} PSAN⁺ progenitors may give rise to Sca^{lo} PSAN⁺ neuroblasts during the early phases of tumour development (week 16 and week 20).

DCX⁺ Neural Progenitors Egress from the SVZ during Tumorigenesis

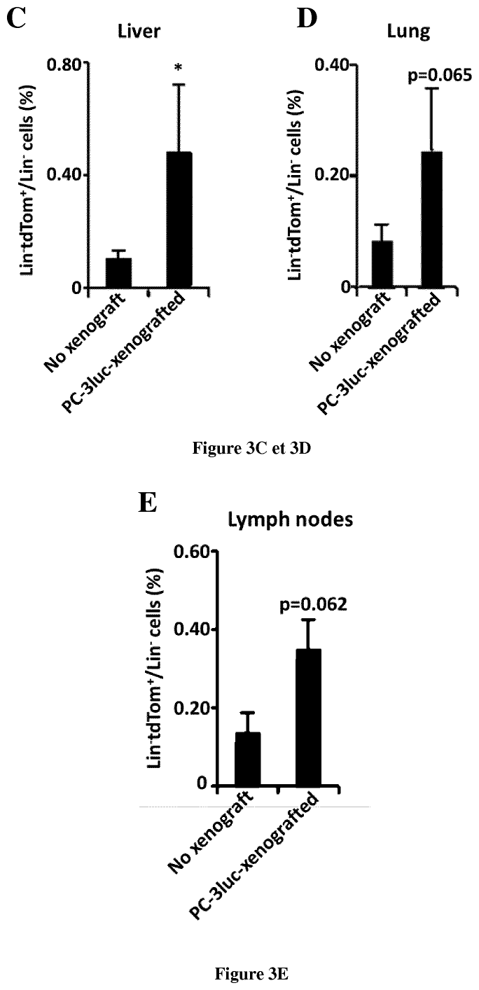

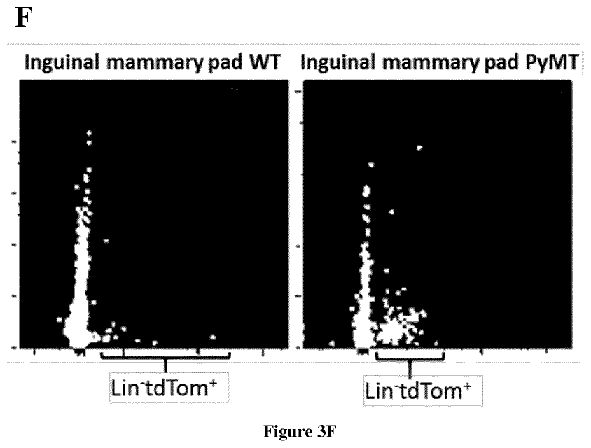

During the development of Hi-Myc prostate cancer, the number of Lin⁻ EYFP⁺ neural precursors in SVZ/OB areas, but not in the DG, oscillated significantly over time (data not shown). These oscillations were not associated with any change of the cellularity of the neurogenic areas of the brain or with any increase of cell death, excluding a non-specific toxicity of tamoxifen (data not shown). Then, we identified 3 sub-populations of Lin⁻ EYFP⁺ neural precursors (Sca⁻ PSAN⁻, Sca^{-/lo} PSAN⁺, Sca^{lo} PSAN^{int}) in the SVZ areas, and found that only the Sca⁻ PSAN⁻ population significantly oscillated during cancer development (data not shown), reaching a low level at week 4, 12 and 20 after birth, when this population was high in the tumour (Sca⁻ PSAN⁻ green population in prostate). Lineage tracing experiments confirmed oscillations of Lin⁻ EYFP⁺ progenitors in the SVZ at 6 weeks after birth, and highlighted the presence of Lin⁻ EYFP⁺ cells in the blood of 6, 12 and 16-week-old Hi-Myc cancer mice (data not shown). These results suggested a potential egress of Lin⁻ EYFP⁺ Sca⁻ PSAN⁻ neural stem/progenitor cells, from the SVZ, that might give rise to Lin⁻ EYFP⁺ Sca⁻ PSAN⁻ neural progenitors homing in the tumour. Further characterizations of Sca⁻ PSAN⁻ populations, in SVZ and tumour, showed that central populations were GFAP⁺ GLAST⁺ neural stem cells while Sca⁻ PSAN⁻ cells in tumours did not express the stem cell markers (data not shown). These results suggested a potential egress of GFAP⁻ GLAST⁻ Lin⁻ EYFP⁺ Sca⁻ PSAN⁻ neural stem cells, from the SVZ, that might give rise to GFAP⁻ GLAST⁻ Lin⁻ EYFP⁺ Sca⁻ PSAN⁻ neural progenitors present in the tumour. In addition, we found CD45⁻ EYFP⁺ cells in the blood of 4-month-old Hi-Myc cancer mice that were not found in healthy littermates (data not shown). To evidence the potential egress, stereotaxic injections of a tdTomato-expressing (tdTom⁺) lentiviral vector into the SVZ of DCX-Cre^{ERT2}/loxp-EYFP/Hi-Myc mice were performed to track precursor cells that could emigrate from the SVZ towards the prostate tumour (data not shown). Lin⁻ Sca⁻ PSAN⁻ tdTom⁺ EYFP⁻ cells were found in the blood at the 4-month tumour stage, indicating the release of this population from the SVZ into the circulation (data not shown), and Lin⁻ tdTom⁺ EYFP⁺ and Lin⁻ tdTom⁺ EYFP⁻ cells were found in the tumour of 5-month-old mice, showing migration of Lin⁻ Sca⁻ PSAN⁻ tdTom⁺ EYFP⁺ and tdTom⁺ EYFP⁻ cells from the SVZ through the bloodstream towards prostate tumours (data not shown) where they could differentiate in neurons (data not shown). Mouse Pten (39) prostate or PyMT (40) breast cancer models underwent similar stereotaxic injections of a tdTomato-expressing lentiviral vector into the SVZ, and displayed a significant accumulation of Lin⁻ tdTom⁺ cells in tumours, indicating that migration of neural progenitors is not restricted to Hi-Myc tumours but might be a more general feature of cancer development (FIGS. 3A and 3F). Finally, we studied various metastatic tissues (colon, liver, lung, lymph nodes) in nude mice orthotopically xenografted with human PC-3luc cancer cells. After injection of the tdTomato-expressing lentiviral vector by stereotaxy into the SVZ, Lin⁻ tdTom⁺ cells were found only in the xenograft and metastatic tissues (Mean number of metastasis: colon (18/mouse), liver (5/mouse), lung (4/mouse) and lymph nodes (5/mouse) by comparison to healthy tissues (FIG. 3B-E), suggesting a selective attraction and migration of neural progenitors in sites colonised by cancer cells to sustain development of metastases.

DCX⁺ Neural Progenitors Regulate Tumour Development

To characterize the role of DCX⁺ progenitors in cancer development, we intercrossed DCX-Cre^{ERT2} mice with an inducible diphtheria toxin receptor (iDTR) line in a nude or Hi-Myc background to study, respectively, tumour growth of luciferase-expressing xenogeneic orthotopic (called later, PC-3luc) or transgenic tumours (data not shown). Selective depletion of DCX⁺ cells after tamoxifen and diphtheria toxin treatment significantly reduced the incidence of neoplastic lesions and inhibited the engraftment of PC-3luc tumour cells, suggesting a critical role for DCX⁺ cells in the early stages of tumour development (data not shown). Further, selective depletion of progenitor cells in the SVZ by stereotaxic injection of diphtheria toxin within the SVZ induced a significant inhibition of tumour development (data not shown). Conversely, orthotopic transplantation of purified Lin⁻ EYFP⁺ cells, isolated from prostate tumour or brain tissues, into established PC-3luc xenografts enhanced tumour growth (data not shown), invasion of lymph nodes (data not shown) and metastasis (data not shown). To study the activity of the 3 sub-populations of neural progenitors (Sca⁻ PSAN⁻, Sca^{lo} PSAN⁺ and Sca^{hi} PSAN⁺) in tumour development and progression, we performed selective depletion experiments in DCX-Cre^{ERT2}/iDTR nude mice before grafting PC-3luc cells mixed with each of the 3 purified sub-populations (data not shown). As the transplantation of activated stem/progenitor cells induces faster kinetics of neuron formation than quiescent cells (28), only the activated Sca^{hi} PSAN⁺ population promoted tumour growth 7 weeks after transplantation (data not shown). These results indicated that DCX⁺ neural progenitors contribute to prostate tumour development and dissemination.

Discussion

Numerous studies have now established that cancer development depends on nerves (4-9). Ingrowth of newly formed autonomic nerve fibres into the tumour contributes to prostate cancer initiation and progression through the respective activation of the β-adrenergic and muscarinic cholinergic signalling (4,7), and also, prostate cancer cells may invade large nerves surrounding the tumour to metastasize, a process called perineural invasion (29). The present study unveils an unprecedented process of tumour-associated neo-neurogenesis by which neural stem/progenitor cells leave the SVZ and reach, through the blood, the primary tumour where they differentiate into neo-nerves that support cancer development and progression. While clinical oncology studies clearly point out the long-term cognitive decline of cancer patients treated by chemotherapies that damage neural progenitor cells in the brain, our study raises the intriguing possibility that the tumour itself might deplete the neurogenic niches in the brain by attracting neural precursor cells to support its own development, and suggest that treatment-naïve cancer patients may, also, develop cognitive impairment (30,31).

The CNS has been shown to regulate the function of peripheral organs, such as leptin-dependent bone formation through modulation of the sympathetic nervous system (32,33), and gut function in healthy and pathological conditions through regulation of the autonomic nervous system (34). Similarly, the CNS can regulate cancer development and progression. Under stress conditions, the CNS can activate the autonomic nervous system or the hypothalamic-pituitary-adrenal axis, and this results to the secretion of divers mediators, such as glucocorticoids and catecholamines, that favour tumour initiation and progression (35). The present study uncovers a novel type of crosstalk between CNS and prostate tumours, as it reveals a unique migration of central neural precursors that nurture tumour development. Together with a recent study that showed that lung tumours could drive distant granulopoiesis in bone, leading to the egress and migration of a neutrophil population to foster the tumour (36), our results also showed how a tumour could have a dialogue with a distant organ to recruit cells that are required for its growth and dissemination. Further studies will be necessary to characterize the molecular events that control the egress of neural stem cells from the brain.

Altogether, these results open new avenues to diagnose and monitor cancer development and to uncover novel therapies targeting neural progenitors in the tumour microenvironment.

### REFERENCES

Throughout this application, various references describe the state of the art to which this invention pertains. The disclosures of these references are hereby incorporated by reference into the present disclosure.

1 Eichmann, A. & Thomas, J. L. Molecular parallels between neural and vascular development. Cold Spring Harb Perspect Med 3, a006551, doi:10.1101/cshperspect.a006551 (2013).

2 Ming, G. L. & Song, H. Adult neurogenesis in the mammalian brain: significant answers and significant questions. Neuron 70, 687-702, doi:10.1016/j.neuron.2011.05.001 (2011).

3 Tanaka, E. M. & Ferretti, P. Considering the evolution of regeneration in the central nervous system. Nature reviews. Neuroscience 10, 713-723, doi:10.1038/nrn2707 (2009).

4 Magnon, C. et al. Autonomic nerve development contributes to prostate cancer progression. Science 341, 1236361, doi:10.1126/science.1236361 (2013).

5 Zhao, C. M. et al. Denervation suppresses gastric tumorigenesis. Science translational medicine 6, 250ra115, doi:10.1126/scitranslmed.3009569 (2014).

6 Pundavela, J. et al. Nerve fibers infiltrate the tumor microenvironment and are associated with nerve growth factor production and lymph node invasion in breast cancer. Molecular oncology 9, 1626-1635, doi:10.1016/j.molonc.2015.05.001 (2015).

7 Dobrenis, K., Gauthier, L. R., Barroca, V. & Magnon, C. Granulocyte colony-stimulating factor off-target effect on nerve outgrowth promotes prostate cancer development. Int J Cancer 136, 982-988, doi:10.1002/ijc.29046 (2015).

8 Hayakawa, Y. et al. Nerve Growth Factor Promotes Gastric Tumorigenesis through Aberrant Cholinergic Signaling. Cancer cell 31, 21-34, doi:10.1016/j.cce11.2016.11.005 (2017).

9 Stopczynski, R. E. et al. Neuroplastic changes occur early in the development of pancreatic ductal adenocarcinoma. Cancer research 74, 1718-1727, doi:10.1158/0008-5472.CAN-13-2050 (2014).

10 Peterson, S. C. et al. Basal cell carcinoma preferentially arises from stem cells within hair follicle and mechanosensory niches. Cell stem cell 16, 400-412, doi:10.1016/j.stem.2015.02.006 (2015).

11 Cohen, S., Levi-Montalcini, R. & Hamburger, V. A Nerve Growth-Stimulating Factor Isolated from Sarcom as 37 and 180. Proceedings of the National Academy of Sciences of the United States of America 40, 1014-1018 (1954).

12 Kobayashi, T., Kihara, K., Hyochi, N., Masuda, H. & Sato, K. Spontaneous regeneration of the seriously injured sympathetic pathway projecting to the prostate over a long period in the dog. BJU Int 91, 868-872 (2003).

13 Ayala, G. E. et al. Cancer-related axonogenesis and neurogenesis in prostate cancer. Clinical cancer research: an official journal of the American Association for Cancer Research 14, 7593-7603 (2008).

14 Francis, F. et al. Doublecortin is a developmentally regulated, microtubule-associated protein expressed in migrating and differentiating neurons. Neuron 23, 247-256 (1999).

15 Couillard-Despres, S. et al. Doublecortin expression levels in adult brain reflect neurogenesis. The European journal of neuroscience 21, 1-14, doi:10.1111/j.1460-9568.2004.03813.x (2005).

16 Brown, J. P. et al. Transient expression of doublecortin during adult neurogenesis. The Journal of comparative neurology 467, 1-10, doi:10.1002/cne.10874 (2003).

17 Zhang, J. & Jiao, J. Molecular Biomarkers for Embryonic and Adult Neural Stem Cell and Neurogenesis. BioMed research international 2015, 727542, doi:10.1155/2015/727542 (2015).

18 Kaplan, M. P., Chin, S. S., Fliegner, K. H. & Liem, R. K. Alpha-internexin, a novel neuronal intermediate filament protein, precedes the low molecular weight neurofilament protein (NF-L) in the developing rat brain. The Journal of neuroscience: the official journal of the Society for Neuroscience 10, 2735-2748 (1990).

19 Schult, D. et al. Expression pattern of neuronal intermediate filament alpha-internexin in anterior pituitary gland and related tumors. Pituitary 18, 465-473, doi:10.1007/s11102-014-0597-2 (2015).

20 Carden, M. J., Trojanowski, J. Q., Schlaepfer, W. W. & Lee, V. M. Two-stage expression of neurofilament polypeptides during rat neurogenesis with early establishment of adult phosphorylation patterns. The Journal of neuroscience: the official journal of the Society for Neuroscience 7, 3489-3504 (1987).

21 Ellwood-Yen, K. et al. Myc-driven murine prostate cancer shares molecular features with human prostate tumors. Cancer cell 4, 223-238 (2003).

22 Walker, T. L., Yasuda, T., Adams, D. J. & Bartlett, P. F. The doublecortin-expressing population in the developing and adult brain contains multipotential precursors in addition to neuronal-lineage cells. The Journal of neuroscience: the official journal of the Society for Neuroscience 27, 3734-3742, doi:10.1523/JNEUROSCI.5060-06.2007 (2007).

23 Lawson, D. A. et al. Basal epithelial stem cells are efficient targets for prostate cancer initiation. Proceedings of the National Academy of Sciences of the United States of America 107, 2610-2615, doi:10.1073/pnas.0913873107 (2010).

24 Ni, J. et al. Epithelial cell adhesion molecule (EpCAM) is associated with prostate cancer metastasis and chemo/radioresistance via the PI3K/Akt/mTOR signaling pathway. The international journal of biochemistry & cell biology 45, 2736-2748, doi:10.1016/j.bioce1.2013.09.008 (2013).

25 Pastrana, E., Cheng, L. C. & Doetsch, F. Simultaneous prospective purification of adult subventricular zone neural stem cells and their progeny. Proceedings of the National Academy of Sciences of the United States of America 106, 6387-6392, doi:10.1073/pnas.0810407106 (2009).

26 Chaker, Z., Codega, P. & Doetsch, F. A mosaic world: puzzles revealed by adult neural stem cell heterogeneity. Wiley interdisciplinary reviews. Developmental biology 5, 640-658, doi:10.1002/wdev.248 (2016).

27 Boutin, C. et al. NeuroD1 induces terminal neuronal differentiation in olfactory neurogenesis. Proceedings of the National Academy of Sciences of the United States of America 107, 1201-1206, doi:10.1073/pnas.0909015107 (2010).

28 Codega, P. et al. Prospective identification and purification of quiescent adult neural stem cells from their in vivo niche. Neuron 82, 545-559, doi:10.1016/j.neuron.2014.02.039 (2014).

29 Liebig, C., Ayala, G., Wilks, J. A., Berger, D. H. & Albo, D. Perineural invasion in cancer: a review of the literature. Cancer 115, 3379-3391, doi:10.1002/cncr.24396 (2009).

30 Schagen, S. B. et al. Monitoring and optimising cognitive function in cancer patients: Present knowledge and future directions. EJC supplements: EJC: official journal of EORTC, European Organization for Research and Treatment of Cancer . . . [et al.] 12, 29-40, doi:10.1016/j.ejcsup.2014.03.003 (2014).

31 Dietrich, J. & Kesari, S. Effect of cancer treatment on neural stem and progenitor cells. Cancer treatment and research 150, 81-95, doi:10.1007/b109924_6 (2009).

32 Ducy, P. et al. Leptin inhibits bone formation through a hypothalamic relay: a central control of bone mass. Cell 100, 197-207 (2000).

33 Takeda, S. et al. Leptin regulates bone formation via the sympathetic nervous system. Cell 111, 305-317 (2002).

34 Rhee, S. H., Pothoulakis, C. & Mayer, E. A. Principles and clinical implications of the brain-gut-enteric microbiota axis. Nature reviews. Gastroenterology & hepatology 6, 306-314, doi:10.1038/nrgastro.2009.35 (2009).

35 Magnon, C. Role of the autonomic nervous system in tumorigenesis and metastasis. Mol Cell Oncol 2, e975643, doi:10.4161/23723556.2014.975643 (2015).

36 Engblom, C. et al. Osteoblasts remotely supply lung tumors with cancer-promoting SiglecF(high) neutrophils. Science 358, doi:10.1126/science.aa15081 (2017).

37 Hagihara, H., Toyama, K., Yamasaki, N. & Miyakawa, T. Dissection of hippocampal dentate gyrus from adult mouse. J Vis Exp, doi:10.3791/1543 (2009).

38 Bemelmans, A. P. et al. Retinal cell type expression specificity of HIV-1-derived gene transfer vectors upon subretinal injection in the adult rat: influence of pseudotyping and promoter. The journal of gene medicine 7, 1367-1374, doi:10.1002/jgm.788 (2005).

39 Wang, S. et al. Prostate-specific deletion of the murine Pten tumor suppressor gene leads to metastatic prostate cancer. Cancer cell 4, 209-221 (2003).

40 Guy, C. T., Cardiff, R. D. & Muller, W. J. Induction of mammary tumors by expression of polyomavirus middle T oncogene: a transgenic mouse model for metastatic disease. Mol Cell Riot 12, 954-961 (1992).

1. A method for predicting the outcome of a patient suffering from prostate cancer or breast cancer comprising the steps of:
i) determining the quantity of DCX⁺ neural progenitor cells in a biological sample obtained from the patient,
ii) comparing the quantity determined at step i) with a corresponding predetermined reference value,
wherein detecting a differential between the quantity determined at step i) and the corresponding predetermined reference value indicates the outcome of the patient.

2. The method of claim 1, wherein the DCX⁺ neural progenitor cells are characterized by expressing at least one of the neural markers selected from the group consisting of Doublecortin (DCX), Polysialylated-neural cell adhesion molecule (PSA-NCAM), Internexin (INA), Sca-1, CD24, EGFR and Nestin.

3. The method of claim 1, comprising a step of concluding that the patient has a good prognosis when the quantity determined at step i) is lower than the corresponding predetermined reference value or concluding that the patient has a poor prognosis when the quantity determined at step i) is higher than the corresponding predetermined reference value.

4. The method of claim 1, comprising a step of concluding that the patient has a non-aggressive, a non-invasive, and/or a non-recurrent prostate cancer when the quantity determined at step i) is lower than the corresponding predetermined reference value or concluding that the patient has an aggressive, an invasive, and/or a recurrent prostate cancer or breast cancer when the quantity determined at step i) is higher than the corresponding predetermined reference value.

5. The method of claim 1, comprising a step of concluding that the patient have a long survival time when the quantity determined at step i) is lower than the corresponding predetermined reference value or concluding that the patient has a short survival time when the quantity determined at step i) is higher than the corresponding predetermined reference value.

6. The method of claim 1, comprising a step of administering an anti-cancer treatment when it is concluded that the patient has a poor prognosis, an aggressive, an invasive, and/or a recurrent prostate cancer or breast cancer, or a short survival time.

7. A method for treating prostate cancer or breast cancer in a patient in need thereof, comprising administering to the patient a therapeutically effective amount of a compound targeting DCX neural progenitor cells.

8. The method of claim 7, wherein the compound targeting DCX⁺ neural progenitor cells is selected from the group consisting of DCX inhibitor, CD24 inhibitor, EGFR inhibitor, Nestin inhibitor, Sca-1 modulator and PSANCAM modulator.

9. The method of claim 7, wherein the the compound targeting DCX⁺ neural progenitor cells is administered in combination with a classical treatment of prostate cancer or breast cancer.

10. A kit or device, comprising means for determining the level of DCX⁺ neural progenitor cells in a biological sample.

11. A kit or device, comprising means for determining at least one of the neural markers selected from the group consisting of Doublecortin (DCX), Polysialylated-neural cell adhesion molecule (PSA-NCAM), Internexin (INA), Sca-1, CD24, EGFR and Nestin.

12. The method of claim 7, wherein the patient has a poor prognosis, an aggressive, an invasive, and/or a recurrent prostate cancer or breast cancer, or a short survival time.
